(19) United States
(12) Patent Application Publication
(10) Pub. No.: US 2015/0132813 A1
(43) Pub. Date: May 14, 2015

(54) BIOSYNTHETIC PATHWAYS, RECOMBINANT CELLS, AND METHODS

(71) Applicant: REGENTS OF THE UNIVERSITY OF MINNESOTA, Minneapolis, MN (US)

(72) Inventors: Kechun Zhang, Minneapolis, MN (US); Yogesh K. Dhande, Minneapolis, MN (US)

(21) Appl. No.: 14/399,681

(22) PCT Filed: Mar. 13, 2013
(86) PCT No.: PCT/US2013/030719

§ 371 Date: Nov. 7, 2014

Related U.S. Application Data
(60) Provisional application No. 61/645,900, filed on May 11, 2012.

(51) Int. Cl. C12P 7/40; C12N 9/02; C12N 9/88

(52) U.S. Cl. CPC C12P 7/40; C12N 9/88; C12N 9/0008; C12Y 401/01; C12Y 102/01003
USPC 435/136; 435/252.3; 435/254.11; 435/471; 435/254.21; 435/254.22; 435/252.33; 435/252.34; 435/252.31

(57) ABSTRACT

This disclosure describes, generally, recombinant cells modified to exhibit increased biosynthesis of pentanoic acid, methods of making such recombinant cells, and methods of inducing the cells to produce pentanoic acid. This disclosure also describes, generally, recombinant cells modified to exhibit increased biosynthesis of 2-methylbutyric acid, methods of making such recombinant cells, and methods of inducing the cells to produce 2-methylbutyric acid.

### CROSS-REFERENCE TO RELATED APPLICATION

This application claims priority to U.S. Provisional Patent Application Ser. No. 61/645,900, filed May 11, 2012, which is incorporated herein by reference.

### SUMMARY

This disclosure describes, in one aspect, a recombinant cell modified to exhibit increased biosynthesis of pentanoic acid compared to a wild-type control. In another aspect, this disclosure describes a recombinant cell modified to exhibit increased biosynthesis of 2-methylbutyric acid compared to a wild-type control.

In each aspect, the recombinant cell can be a fungal cell or a bacterial cell. In each aspect, the recombinant cell can be photosynthetic. In each aspect, the recombinant cell can be cellulolytic.

In the aspect in which the recombinant cell exhibits increased biosynthesis of pentanoic acid, the increased biosynthesis of pentanoic acid can include an increase in conversion of L-aspartate to L-threonine compared to a wild-type control, an increase in conversion of L-threonine to 2-ketobutyrate compared to a wild-type control, an increase in 2-ketobutyrate elongation activity compared to a wild-type control, an increase in 2-ketovalerate elongation activity compared to a wild-type control, an increase in ketoacid decarboxylase activity compared to a wild-type control, an increase in ketoacid decarboxylase selectivity toward a predetermined substrate compared to a wild-type control, or an increase in aldehyde dehydrogenase activity compared to a wild-type control.

In the aspect in which the recombinant cell exhibits increased biosynthesis of 2-methylbutyric acid, the increased biosynthesis of 2-methylbutyric acid can include an increase in conversion of L-aspartate to L-threonine compared to a wild-type control, an increase in conversion of L-threonine to 2-ketobutyrate compared to a wild-type control, an increase in conversion of 2-ketobutyrate to 2-keto-3-methylvalerate, an increase in ketoacid decarboxylase activity compared to a wild-type control, an increase in ketoacid decarboxylase selectivity toward a predetermined substrate compared to a wild-type control, or an increase in aldehyde dehydrogenase activity compared to a wild-type control.

In another aspect, this disclosure describes a method that generally includes incubating a recombinant cell that exhibits increased biosynthesis of pentanoic acid in medium that includes a carbon source under conditions effective for the recombinant cell to produce pentanoic acid. In some embodiments, the carbon source can include one or more of: glucose, pyruvate, L-aspartate, L-threonine, 2-ketobutyrate, 2-ketovalerate, 2-ketocaproate, valeraldehyde, CO₂, cellulose, xylose, sucrose, arabinose, or glycerol.

In another aspect, this disclosure describes a method that generally includes incubating a recombinant cell that exhibits increased biosynthesis of 2-methylbutyric acid in medium that includes a carbon source under conditions effective for the recombinant cell to produce 2-methylbutyric acid. In some embodiments, the carbon source can include one or more of: glucose, pyruvate, L-aspartate, L-threonine, 2-ketobutyrate, 2-keto-3-methylvalerate, 2-methyl butyraldehyde, CO₂, cellulose, xylose, sucrose, arabinose, or glycerol.

In another aspect, this disclosure describes a method that generally includes introducing into a host cell a heterologous polynucleotide encoding at least one polypeptide that catalyzes conversion of a carbon source to pentanoic acid, wherein the at least one polynucleotide is operably linked to a promoter so that the modified host cell catalyzes conversion of the carbon source to pentanoic acid.

In another aspect, this disclosure describes a method that generally includes introducing into a host cell a heterologous polynucleotide encoding at least one polypeptide that catalyzes conversion of a carbon source to 2-methylbutyric acid, wherein the at least one polynucleotide is operably linked to a promoter so that the modified host cell catalyzes conversion of the carbon source to 2-methylbutyric acid.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Routes for production of 2-methylbutyric acid (2 MB) and pentanoic acid (PA). (A) Chemical process for 2-methylbutyric acid and pentanoic acid from 1-Butene and 2-Butene. (B) Metabolic pathway for synthesis of 2-methylbutyric acid from glucose. (C) Metabolic pathway for synthesis of pentanoic acid from glucose.

FIG. 2. Synthetic operons for (A) 2-methylbutyric acid (2 MB) production. (B) Pentanoic acid (PA) production. DC, 2-ketoacid decarboxylase; DH, aldehyde dehydrogenase.

FIG. 3. Results of fermentation experiments with different aldehyde dehydrogenases. (A) Comparison of aldehyde dehydrogenases for 2-methylbutyric acid production. (B) Comparison of aldehyde dehydrogenases for production of pentanoic acid.

FIG. 4. Results of fermentation experiments with different ketoacid decarboxylases. (A) Comparison of ketoacid decarboxylases for 2-methylbutyric acid production. (B) Comparison of ketoacid decarboxylases for production of pentanoic acid.

FIG. 5. Results of fermentation experiments for combinations of ketoacids decarboxylases and aldehyde dehydrogenases (A) Comparison of various combinations for 2-methylbutyric acid production. (B) Comparison of various combinations for production of pentanoic acid.

### DETAILED DESCRIPTION OF ILLUSTRATIVE EMBODIMENTS

In the description of exemplary embodiments that follow, certain metabolic enzymes, and the natural source of those enzymes, are specified. These are merely examples of suitable enzymes and suitable sources of the specified enzymes. Alternative enzymes with similar catalytic activities are possible, as are homologs that are obtainable from different microbial species or strains. Accordingly, the exemplary embodiments described herein should not be construed as limiting the scope of the microbes or methods that are reflected in the claims.

Pentanoic acid and 2-methylbutyric acid serve as chemical intermediates for a variety of applications such as, for example, plasticizers, lubricants, and pharmaceuticals. This disclosure describes the construction of synthetic metabolic pathways in Escherichia coli to biosynthesize these two acids: the native leucine biosynthetic pathway was modified to produce pentanoic acid; the native isoleucine biosynthetic pathway was modified to produce 2-methylbutyric acid. Various aldehyde dehydrogenases and 2-ketoacid decarboxylases were investigated for their activities in the constructed pathways. Highest titers of 2.59 g/L for 2-methylbutyric acid and 2.58 g/L for pentanoic acid were achieved through optimal combinations of enzymes in shake flask fermentation. This work demonstrates the feasibility of renewable production of high volume aliphatic acids.

Crude oil is a major source of energy and industrial organic chemicals. However, crude oil reserves are being actively depleted making the development of sustainable routes to fuels and chemicals more attractive. To address this challenge, one can take a biosynthetic approach involving engineering microbes to produce non-natural chemical intermediates. Production of non-natural metabolites can involve the engineering and development of synthetic metabolic pathways. In this work, biosynthetic strategies were developed for renewable production of pentanoic acid (PA) and 2-methylbutyric acid (2 MB) from glucose or other suitable carbon source.

The total U.S. consumption of pentanoic acid and 2-methylbutyric acid was approximately 14,000 metric tons in 2005 (Dow. Product Safety Assessment: Isopentanoic Acid. The Dow chemical company 2008). These chemicals can serve as intermediates for a variety of applications such as plasticizers, lubricants, and pharmaceuticals. They are also used for extraction of mercaptans from hydrocarbons. Esters of pentanoic acid are gaining increased attention as pentanoic biofuels because they can be used in both gasoline and diesel with very high blend ratios (Lange et al., Angew Chem Int Edit 2010; 49:4479-4483). Commercially, these chemicals are typically manufactured by oxidizing valeraldehyde and/or 2-methyl butyraldehyde, each of which may be made through a process in which a petroleum-based compound is reacted with synthesis gas (Dow. Product Safety Assessment: Isopentanoic Acid. The Dow chemical company 2008). Since the process uses toxic intermediates like synthesis gas and non-renewable petroleum-based feedstock, a sustainable route to these chemicals is needed. Biosynthesis is presented here as a potential alternative route to these chemicals.

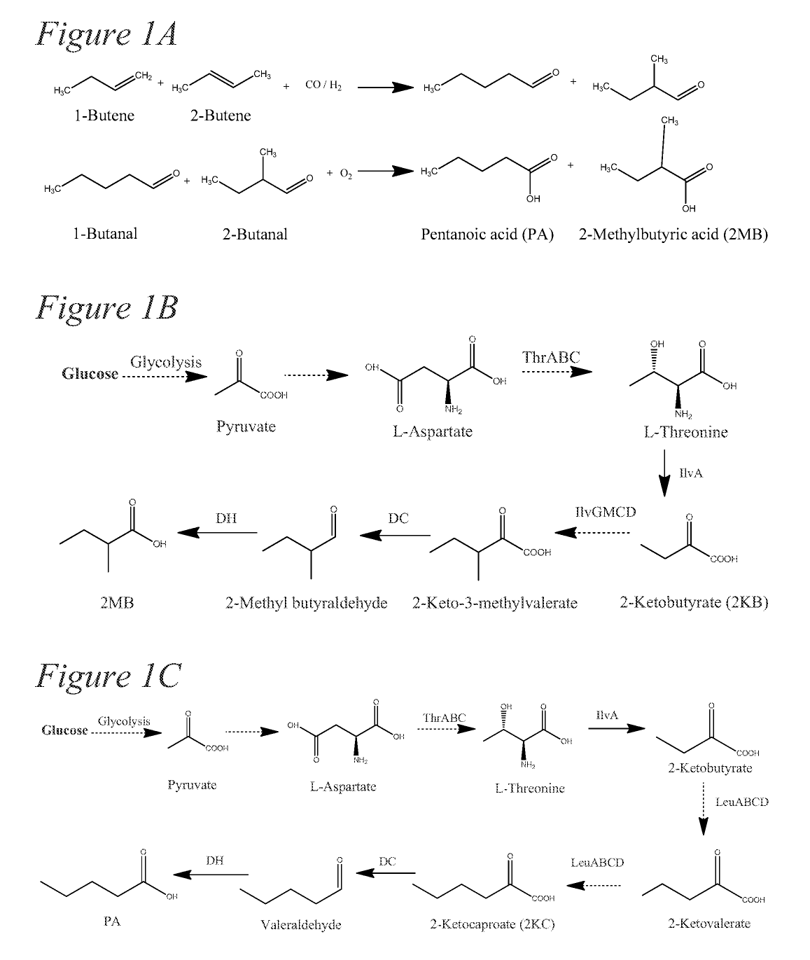

One advantage of engineered biosynthetic pathways is the conservation of native biosynthetic pathways between microbes. Thus, once a newly engineered biosynthetic pathway is established in one microbe, it often can be employed in other microbes. In this work, the native leucine and isoleucine biosynthetic pathways in E. coli were modified by introducing into the E. coli host cells heterologous (non-native) enzymes aldehyde dehydrogenase and/or 2-ketoacid decarboxylase. Exemplary synthetic metabolic routes to 2-methylbutyric acid and pentanoic acid are shown in FIG. 1B and FIG. 1C, respectively. A common intermediate for both the pathways is 2-ketobutyrate (2 KB), which is derived from threonine by biosynthetic deaminase IlvA. Overexpressing thrA, thrB, and thrC can drive the carbon flux towards threonine biosynthesis (Zhang et al., Proc Natl Acad Sci USA 2010; 107:6234-6239) and, therefore, into the synthetic metabolic pathways to produce pentanoic acid and/or 2-methylbutyric acid.

For the synthesis of 2-methylbutyric acid, shown in FIG. 1B, 2-ketobutyrate is driven into synthesis of 2-keto-3-methylvalerate (KMV), the penultimate precursor to 2-methylbutyric acid. The condensation of 2-ketobutyrate and pyruvate to 2-aceto-2-hydroxybutyrate (AHB) is catalyzed by IlvG and IlvM. Another two enzymes IlvC and IlvD can catalyze conversion of AHB into KMV. KMV is then decarboxylated by a ketoacid decarboxylase (DC) into 2-methyl butyraldehyde, which can be oxidized to 2-methylbutyric acid by an aldehyde dehydrogenase (DH).

For the synthesis of pentanoic acid, 2-ketobutyrate can undergo two cycles of “+1” carbon chain elongation to make 2-ketocaproate (2KC). In the native leucine biosynthetic pathway, 2-ketoisovalerate is converted to 2-ketoisocaproate through a 3-step chain elongation cycle catalyzed by 2-isopropylmalatesynthase (LeuA), isopropyl malate isomerase complex (LeuC, LeuD) and 3-isopropylmalate dehydrogenase (LeuB). In our synthetic pathways, however, LeuA, LeuB, LeuC, and LeuD are flexible enough to similarly elongate 2-ketobutyrate to 2-ketovalerate, and then to elongate 2-ketovalerate to 2-ketocaproate (4). 2-ketocaproate can then be decarboxylated by a 2-ketoacid decarboxylase (DC) into valeraldehyde, which can be oxidized to pentanoic acid by a dehydrogenase (DH).

### Construction of Metabolic Pathways for Biosynthesis of 2-Methylbutyric Acid and Pentanoic Acid

The biosynthetic schemes for the production of 2-methylbutyric acid (2 MB) and pentanoic acid (PA) are shown in FIG. 1B and FIG. 1C, respectively. All enzymes downstream of aspartate biosynthesis were overexpressed from three synthetic operons. One operon includes coding regions for ThrA, ThrB, and ThrC, each of which is involved in threonine synthesis, under control of the P_{L}lacO1 promoter on a low copy plasmid pIPA1 carrying spectinomycin resistance marker. For 2-methylbutyric acid synthesis (FIG. 1B), ilvA, ilvG, ilvM, ilvC, and ilvD were cloned on a low copy plasmid with a kanamycin resistance marker to get pIPA2. Similarly, for synthesis of pentanoic acid, ilvA, leuA, leuB, leuC, and leuD were cloned on a low copy plasmid pIPA3 carrying a kanamycin resistance marker. Various aldehyde dehydrogenases and ketoacid decarboxylases were present under P_{L}lacO1 promoter in the transcriptional order DC-DH (2-ketoacid decarboxylase-dehydrogenase) on high copy plasmids (pIPA4 to pIPA15, Table 2) carrying ampicillin resistance marker.

Since threonine is a common intermediate in both the pathways, a threonine overproducer E. coli strain ATCC98082 was used in the study. The strain had threonine exporter gene rhtA removed to ensure high intracellular level of threonine (Zhang et al., Proc Natl Acad Sci USA 2010; 107:6234-6239) as well as the alcohol dehydrogenase yqhD gene deletion to eliminate the side reactions leading to respective alcohols. The resultant strain is referred to hereafter as the PA1 strain.

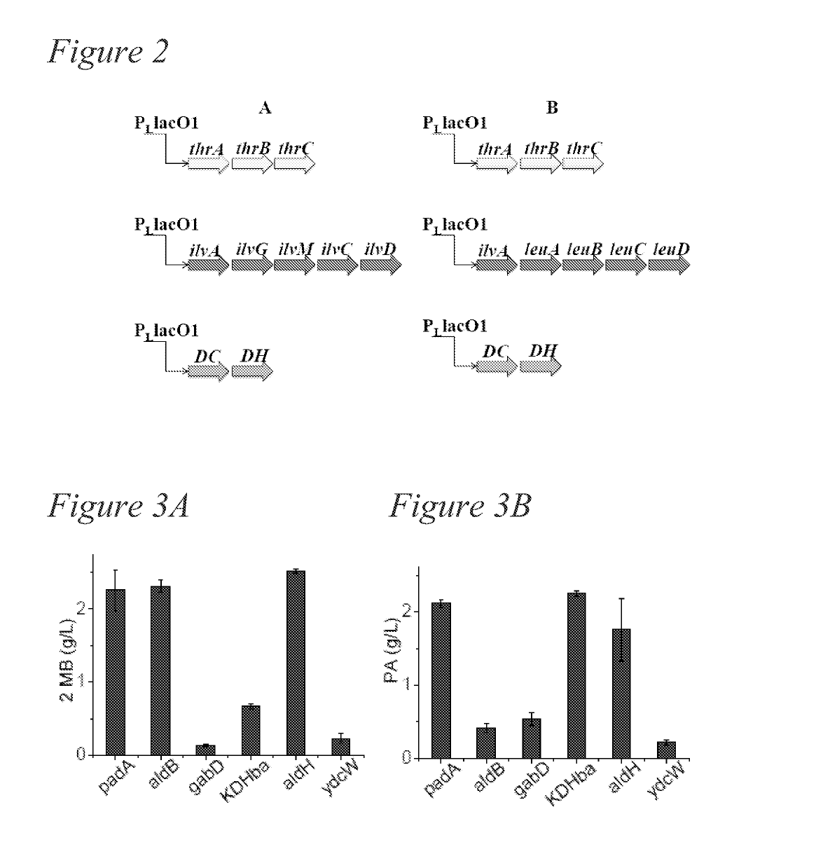

The synthetic pathways shown in FIG. 1B and FIG. 1C were designed to include decarboxylation of ketoacids 2-keto-3-methylvalerate (FIG. 1B) and 2-ketocaproate (2KC, FIG. 1C) into their respective aldehydes, followed by oxidation of the aldehydes to carboxylic acids. Based on our previous work on producing isobutyric acid (Zhang et al., ChemSusChem 2011; 4:1068-1070), we cloned wild-type 2-ketoisovalerate decarboxylase KIVD from Lactococcus lactis (de la Plaza et al., FEMS Microbiol Lett 2004; 238:367-374) and phenylacetaldehyde dehydrogenase PadA (Rodriguez-Zavala et al., Protein Sci 2006; 15:1387-1396) from E. coli to check the production of our target chemicals. The PA1 strain was transformed with plasmids pIPA1, pIPA2, and pIPA4 to produce 2-methylbutyric acid. The PA1 strain was transformed with plasmids pIPA1, pIPA3, and pIPA4 to produce pentanoic acid. Shake flask fermentations were carried out using each recombinant strain. Using this approach, we produced 2.26 g/L of 2-methylbutyric acid and 2.12 g/L of pentanoic acid, demonstrating the feasibility of our biosynthetic approach.

### Screening of Aldehyde Dehydrogenases

In order to improve production titers, the effect of choosing different aldehyde dehydrogenases was examined (FIG. 3A and FIG. 3B). Six aldehyde dehydrogenases were selected as candidate enzymes for this study: acetaldehyde dehydrogenase AldB (Ho and Weiner, J Bacteriol 2005; 187:1067-1073), 3-hydroxypropionaldehyde dehydrogenase AldH (Jo et al., Appl Microbiol Biotechnol 2008; 81:51-60), phenylacetaldehyde dehydrogenase PadA (Rodríguez-Zavala et al., Protein Sci 2006; 15:1387-1396), succinate semialdehyde dehydrogenase GabD (Bartsch et al., J Bacteriol 1990; 172:7035-7042), γ-aminobutyraldehyde dehydrogenase YdcW (Gruez et al., J Mol Biol 2004; 343:29-41) from E. coli, and α-ketoglutaric semialdehyde dehydrogenase KDH_{ba} (Jo et al., Appl Microbiol Biotechnol 2008; 81:51-60) from Burkholderia ambifaria. Bacterial strains were constructed with three synthetic operons as shown in FIG. 2. All heterologous enzymes introduce into the strains were identical across the strain with the exception of the aldehyde dehydrogenase. Wild-type KIVD was selected as the 2-ketoacid decarboxylase for each strain Shake flask fermentations were carried out at 30° C. and samples were analyzed by HPLC. The fermentations were analyzed to identify the strain—and therefore the aldehyde dehydrogenase—that produced the highest quantity of desired product.

To compare activities of various aldehyde dehydrogenases for producing 2-methylbutyric acid, the PA1 strain was transformed with plasmids pIPA1, pIPA2, and any one of pIPA4 to pIPA9. After fermentation, the highest titer of 2.51 g/L was achieved with AldH, while Al dB, PadA, KDH_{ba}, GabD and YdcW produced 2.31 g/L, 2.26 g/L, 0.67 g/L, 0.14 g/L and 0.23 g/L, respectively (FIG. 3A).

For production of pentanoic acid, the PA1 strain was transformed with plasmids pIPA1, pIPA3, and any one of pIPA4 to pIPA9. KDH_{ba} was found to be most active aldehyde dehydrogenase for producing pentanoic acid (2.25 g/L), while AldH, Al dB, PadA, GabD and YdcW produced 1.76 g/L, 0.42 g/L, 2.12 g/L, 0.54 g/L, and 0.22 g/L, respectively (FIG. 3B).

### Screening of 2-Ketoacid Decarboxylases

Several metabolic byproducts such as acetate, propionic acid, butyric acid, and 3-methylbutyric acid were observed during fermentation. Wild-type ketoacid decarboxylase KIVD from Lactococcus lactis (de la Plaza et al., FEMS Microbiol Lett 2004; 238:367-374) and several of its mutants were investigated for an increase in yield of target C5 acids and a reduction in byproduct formation. The single amino acid substitution mutation V461A was reported to increase the specificity of KIVD towards larger substrates. The effect of three other mutations M538A, F381L, and F542L, each in combination with the V461A mutation, was investigated. These mutations replace a bulky residue in key locations by a smaller hydrophobic residue. The effect of indolepyruvate decarboxylase (IPDC) from Salmonella typhimurium also was studied. Plasmids were constructed with different 2-ketoacid decarboxylases but all possessed the same aldehyde dehydrogenase (PadA) and other enzymes.

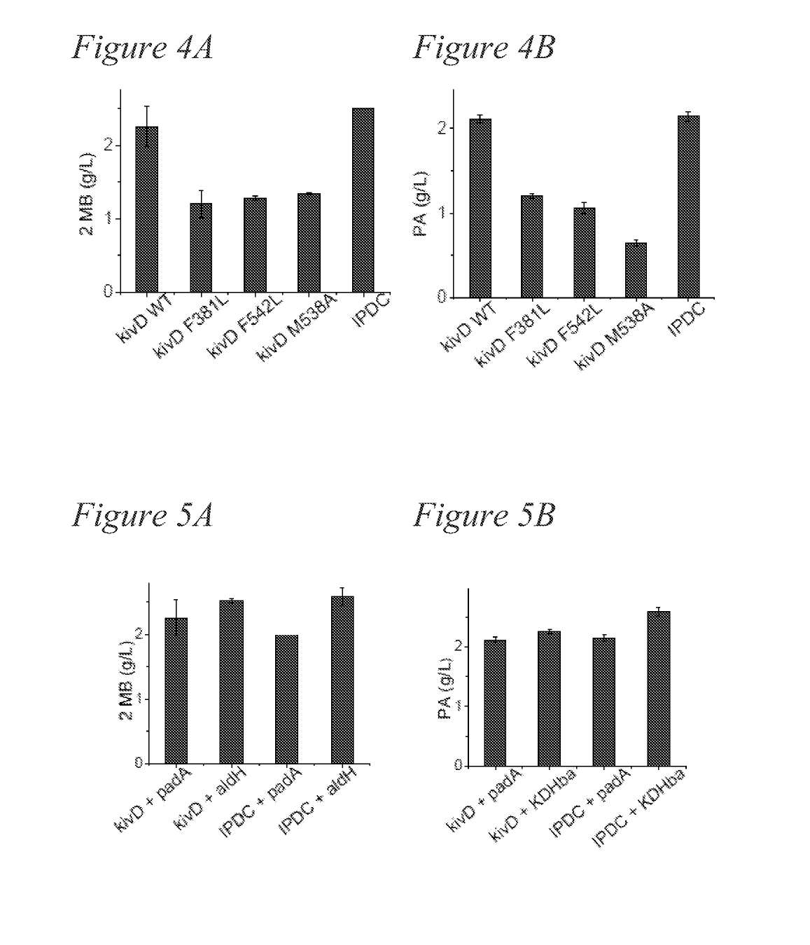

To compare the activities of the selected 2-ketoacid decarboxylases for 2-methylbutyric acid synthesis, the PA1 strain was transformed with pIPA1, pIPA2, and any one of the plasmids pIPA10 to pIPA13 for 2-methylbutyric acid. To compare the activities of the selected 2-ketoacid decarboxylases for pentanoic acid synthesis, the PA1 strain was transformed with pIPA1, pIPA3, and any one of the plasmids pIPA10 to pIPA13 for pentanoic acid synthesis. Shake flask fermentations showed that IPDC worked better than KIVD or any of its mutants for producing either 2-methylbutyric acid (2.5 g/L) or pentanoic acid (2.14 g/L). (FIG. 4A and FIG. 4B).

Having established that AldH and IPDC have the highest activity among all of candidate aldehyde dehydrogenases and 2-ketoacid decarboxylases for the production of 2-methylbutyric acid, they were combined together (pIPA14) to investigate if the effects are additive. In combination, 2-methylbutyric acid titer reached 2.59 g/L, only marginally higher than 2.51 g/L for AldH with WT KIVD or 2.5 g/L for PadA with IPDC (FIG. 5A). Similarly, KDH_{ba} was cloned together with IPDC (pIPA15) for the production of pentanoic acid. This increased pentanoic acid titer to 2.58 g/L. In comparison, the production titer was 2.25 g/L for KDH_{ba} with WT KIVD or 2.14 g/L for PadA with IPDC (FIG. 5B).

### Purification and Characterization of Enzymes

The most active ketoacid decarboxylase, IPDC, and most active aldehyde dehydrogenases, AldH and KDH_{ba}, were characterized for their activity on substrates involved in the constructed pathways. AldH was expressed from a His-tag plasmid and purified. IPDC and KDH_{ba} were available from earlier study. The kinetic parameters were measured by monitoring the NADH absorbance at 340 nm. The values for k_{cat} and K_{M} are given in Table 1.

TABLE 1
Kinetic Parameters for Enzymes

| Enzyme | Substrate | K_{M} (mM) | k_{cat} (s⁻¹) | k_{cat}/K_{M} |
|---|---|---|---|---|
| IPDC | 2-Keto-3-methylvalerate | 0.85 ± 0.18 | 4.13 ± 0.21 | 4.86 |
| AldH | 2-Methyl butyraldehyde | 1.89 ± 0.24 | 3.55 ± 0.17 | 1.88 |
| IPDC | 2-Ketocaproate | 0.63 ± 0.1 | 1.89 ± 0.06 | 3 |
| KDH_{ba} | Valeraldehyde | 0.031 ± 0.005 | 8.69 ± 0.26 | 289.7 |

In vitro enzymatic assays were carried out to confirm that these enzymes indeed have good activities towards target substrates. The kinetic parameters were measured by monitoring the NADH absorbance at 340 nm. The activity of IPDC was measured using a coupled enzymatic assay method. The values for the catalytic rate constant (k_{cat}) and Michaelis-Menten constant (K_{M}) are given in Table 1. The K_{M} and k_{cat} of IPDC for 2-keto-3-methylvalerate were determined to be 0.85 mM and 4.13 s⁻¹, while the K_{M} and k_{cat} for 2-ketocaproate were 0.63 mM and 1.89 s⁻¹ respectively. The specificity constants k_{cat}/K_{M} of IPDC for both the substrates were found to be very close. The K_{M} and k_{cat} of AldH for 2-methyl butyraldehyde were found to be 1.89 mM and 3.55 s⁻¹. KDH_{ba} has significantly lower K_{M} towards valeraldehyde (0.031 mM) than smaller or branched substrates like isobutyraldehyde (34.5 mM) and isovaleraldehyde (7.62 mM) but similar k_{cat} values (Xiong et al., Sci Rep 2012; 2). Therefore, the specificity constant (k_{cat}/K_{M}) of KDH_{ba} towards valeraldehyde is 1260-fold and 308-fold higher than those toward isobutyraldehyde and isovaleraldehyde.

Pentanoic acid and 2-methylbutyric acid are two valuable chemical intermediates in chemical industry. The purpose of this study was to investigate feasibility of biosynthetic approach to synthesize these chemicals. We were successful in modifying the native leucine and isoleucine biosynthetic pathways to produce these non-natural chemicals in E. coli. The heterologous enzymes involved in the pathways were overexpressed by cloning polynucleotides that encode the enzymes into a synthetic operon. The designed pathways exemplified herein include decarboxylation of ketoacids 2-keto-3-methylvalerate and 2-ketocaproate into respective aldehydes, followed by oxidation to carboxylic acids. In this work, we investigated these last two steps to improve production quantities. We cloned wild-type kivD and padA to investigate production of our target chemicals. We observed production levels of 2.26 g/L for 2-methylbutyric acid and 2.12 g/L for pentanoic acid from 40 g/L glucose after two days of shake flask fermentation. This confirmed the feasibility of our biosynthetic approach.

In order to improve the product titers, we then examined the effect of different aldehyde dehydrogenases on product titers in shake flask fermentation. KDH_{ba} was found to be the most effective aldehyde dehydrogenase among those examined for production of pentanoic acid. AldH proved most effective aldehyde dehydrogenase among those examined for 2-methylbutyric acid production.

Several byproducts such as propionic acid, butyric acid, and 3-methylbutyric acid were observed during the fermentation to produce 2-methylbutyric acid or pentanoic acid. We therefore sought to further increase production of our target products by directing biosynthesis away from these byproducts and toward 2-methylbutyric acid or pentanoic acid. Mutants of KivD were shown previously to increase the decarboxylation activity towards larger ketoacid substrates (Bartsch et al., J Bacteriol 1990; 172:7035-7042). Thus, we compared KivD to several KivD mutants and IPDC for their ability to increase production of target compounds by reducing the byproducts. IPDC was most effective at directing biosynthesis away from undesired byproducts and toward the desired compounds. Thus, we were able to achieve a production titer of 2.59 g/L for 2-methylbutyric acid with IPDC-AldH and a production titer of 2.58 g/L for pentanoic acid with IPDC-KDH_{ba}. This production corresponds to yields of 22.1% and 16.6% of theoretical maximum (0.28 g/g of glucose and 0.38 g/g of glucose) for pentanoic acid and 2-methylbutyric acid respectively. Finally, enzymatic assays were carried out to confirm the activities of these enzymes and to find the kinetic parameters.

This work demonstrates the feasibility of renewable production of these chemicals. To the best of our knowledge, this is the earliest report of metabolic engineering for the synthesis of C5 monocarboxylic acids. This work also demonstrates the use of aerobic process for production of acids. The organisms capable of producing acids typically do so in anaerobic conditions, which also results in significant acetate production, thus reducing the yield from glucose. Use of aerobic process will allow better control and reduce the acetate levels in fermentation broths. This can be accomplished in a carefully operated stirred-tank type fermenter where oxygen is provided by passing air through the tank. Such fermenters will also able to achieve high cell densities, which can lead to greater production of the desired product compounds.

The biosynthetic strategy described herein is a promising advance towards sustainable production of such platform chemicals. Moreover, since the biosynthetic pathways described herein are modifications of the host's native amino acid biosynthetic pathways, and those native biosynthetic pathways are highly conserved across species, the biosynthetic modifications described herein may be applied to the native biosynthetic pathways of a variety of additional organisms.

Thus, in one aspect, the invention provides recombinant microbial cell modified to exhibit increased biosynthesis of pentanoic acid compared to a wild-type control. In another aspect, the invention provides a recombinant microbial cell modified to exhibit increased biosynthesis of 2-methylbutyric acid compared to a wild-type control. In some cases, the wild-type control may be unable to produce pentanoic acid or 2-methylbutyric acid and, therefore, an increase in the biosynthesis of a particular product may reflect any measurable biosynthesis of that product. In certain embodiments, an increase in the biosynthesis of pentanoic acid or 2-methylbutyric acid can include biosynthesis sufficient for a culture of the microbial cell to accumulate pentanoic acid or 2-methylbutyric acid to a predetermine concentration.

The predetermined concentration may be any predetermined concentration of the product suitable for a given application. Thus, a predetermined concentration may be, for example, a concentration of at least 0.1 g/L such as, for example, at least 0.5 g/L, at least 1.0 g/L, at least 2.0 g/L, at least 3.0 g/L, at least 4.0 g/L, at least 5.0 g/L, at least 6.0 g/L, at least 7.0 g/L, at least 8.0 g/L, at least 9.0 g/L, at least 10 g/L, at least 20 g/L, at least 50 g/L, at least 100 g/L, or at least 200 g/L.

The recombinant cell can be, or be derived from, any suitable microbe including, for example, a prokaryotic microbe or a eukaryotic microbe. As used herein, the term “or derived from” in connection with a microbe simply allows for the “host cell” to possess one or more genetic modifications before being modified to exhibit the indicated increased biosynthetic activity. Thus, the term “recombinant cell” encompasses a “host cell” that may contain nucleic acid material from more than one species before being modified to exhibit the indicated biosynthetic activity. As noted above, the leucine and isoleucine biosynthetic pathways that are the basis for our engineered biosynthetic pathways are highly conserved across species. This conservation across species means that our pathways, exemplified in an E. coli host, may be introduced into other host cell species, if desired.

In some embodiments, the host cell may be selected to possess one or more natural physiological activities. For example, the host cell may be photosynthetic (e.g., cyanobacteria) or may be cellulolytic (e.g., Clostridium cellulolyticum).

In some embodiments, the recombinant cell may be, or be derived from, a eukaryotic microbe such as, for example, a fungal cell. In some of these embodiments, the fungal cell may be, or be derived from, a member of the Saccharomycetaceae family such as, for example, Saccharomyces cerevisiae, Candida rugosa, or Candida albicans.

In other embodiments, the recombinant cell may be, or be derived from, a prokaryotic microbe such as, for example, a bacterium. In some of these embodiments, the bacterium may be a member of the phylum Protobacteria. Exemplary members of the phylum Protobacteria include, for example, members of the Enterobacteriaceae family (e.g., Escherichia coli) and, for example, members of the Pseudomonaceae family (e.g., Pseudomonas putida). In other cases, the bacterium may be a member of the phylum Firmicutes. Exemplary members of the phylum Firmicutes include, for example, members of the Bacillaceae family (e.g., Bacillus subtilis), members of the Clostridiaceae family (e.g., Clostridium cellulolyticum) and, for example, members of the Streptococcaceae family (e.g., Lactococcus lactis). In other cases, the bacterium may be a member of the phylum Cyanobacteria.

In some embodiments, the increased biosynthesis of pentanoic acid compared to a wild-type control can include an increase in elongating 2-ketobutyrate to 2-ketovalerate compared to a wild-type control, an increase in elongating 2-ketovalerate to 2-ketocaproate compared to wild-type control, increased ketoacid decarboxylase activity compared to a wild-type control, and/or increased aldehyde dehydrogenase activity compared to a wild-type control. In other embodiments, the increased biosynthesis of 2-methylbutyric acid compared to a wild-type control can include increased conversion of threonine to 2-ketobutyrate compared to a wild-type control, increased conversion of 2-ketobutyrate to 2-keto-3-methylvalerate compared to a wild-type control, increased ketoacid decarboxylase activity compared to a wild-type control, and/or increased aldehyde dehydrogenase activity compared to a wild-type control. In some cases, at least a portion of the increased ketoacid decarboxylase activity can result from modification of the ketoacid decarboxylase enzyme. For example, 2-ketoacid decarboxylase of Lactococcus lactis (or an analog) may be modified to include at least one amino acid substitution selected from: V461A, M538A, or F542L, or an analogous substitution. In some cases, the 2-ketoacid decarboxylase can be modified to include the V461A substitution (or an analogous substitution) in combination with either the M528A substitution (or an analogous substitution) or the V461A substitution (or an analogous substitution).

As used herein, the term “analog” refers to a related enzyme from the same or a different microbial source with similar enzymatic activity. As such, analogs often show significant conservation and it is a trivial matter for a person of ordinary skill in the art to identify a suitable related analog of any given enzyme. Also, it is a trivial matter for a person of ordinary skill in the art to identify an “analogous substitution” by aligning the amino acid sequence of the analog with the amino acid sequence of the reference enzyme. Thus, positional differences and/or amino acid residue differences may exist between the recited substitution and an analogous substitution despite conservation between the analog and the reference enzyme.

In some embodiments, the recombinant cell can exhibit an increase in indolepyruvate decarboxylase (IPDC) activity. The increase in IPDC activity can result from expression of an IPDC enzyme. Exemplary IPDC enzymes include, for example, any one of the polypeptides reflected in any one of SEQ ID NO:1-21. Thus, in some embodiments, the recombinant cell can include a heterologous polynucleotide sequence that encodes an IPDC decarboxylase such as, for example, any one of the polypeptides reflected in any one SEQ ID NO:1-21.

In some embodiments the recombinant cell can exhibit an increase in aldehyde dehydrogenase activity. The increase in aldehyde dehydrogenase activity can result from expression of an aldehyde dehydrogenase enzyme. Exemplary aldehyde dehydrogenase enzymes include, for example, any one of the polypeptides reflected in any one of SEQ ID NO:22-55. Thus, in some embodiments, the recombinant cell can include a heterologous polynucleotide sequence that encodes an aldehyde dehydrogenase such as, for example, any one of the polypeptides reflected in any one SEQ ID NO:22-55.

As used herein, the term “activity” with regard to particular enzyme refers to the ability of a polypeptide, regardless of its common name or native function, to catalyze the conversion of the enzyme's substrate to a product, regardless of whether the “activity” is less than, equal to, or greater than the native activity of the identified enzyme. Methods for measuring the biosynthetic activities of cells are routine and well known to those of ordinary skill in the art. In the context of a genetically-modified cell, the term “activity” refers to the ability of the genetically-modified cell to synthesize an identified product compound, regardless of whether the “activity” is less than, equal to, or greater than the native activity of a wild-type strain of the cell.

As used herein, an increase in catalytic activity of an enzyme or an increase in the biosynthetic activity of a genetically-modified cell can be quantitatively measured and described as a percentage of the activity of an appropriate wild-type control. The catalytic activity exhibited by a genetically-modified polypeptide or the biosynthetic activity of a genetically-modified cell can be, for example, at least 110%, at least 125%, at least 150%, at least 175%, at least 200% (two-fold), at least 250%, at least 300% (three-fold), at least 400% (four-fold), at least 500% (five-fold), at least 600% (six-fold), at least 700% (seven-fold), at least 800% (eight-fold), at least 900% (nine-fold), at least 1000% (10-fold), at least 2000% (20-fold), at least 3000% (30-fold), at least 4000% (40-fold), at least 5000% (50-fold), at least 6000% (60-fold), at least 7000% (70-fold), at least 8000% (80-fold), at least 9000% (90-fold), at least 10,000% (100-fold), or at least 100,000% (1000-fold) of the activity of an appropriate wild-type control.

Alternatively, an increase in catalytic activity may be expressed as an increase in k_{cat} such as, for example, at least a two-fold increase, at least a three-fold increase, at least a four-fold increase, at least a five-fold increase, at least a six-fold increase, at least a seven-fold increase, at least an eight-fold increase, at least a nine-fold increase, at least a 10-fold increase, at least a 15-fold increase, or at least a 20-fold increase in the k_{cat} value of the enzymatic conversion.

An increase in catalytic activity also may be expressed in terms of a decrease in Kₘ such as, for example, at least a two-fold decrease, at least a three-fold decrease, at least a four-fold decrease, at least a five-fold decrease, at least a six-fold decrease, at least a seven-fold decrease, at least an eight-fold decrease, at least a nine-fold decrease, at least a 10-fold decrease, at least a 15-fold decrease, or at least a 20-fold decrease in the Kₘ value of the enzymatic conversion.

A decrease in catalytic activity of an enzyme or an increase in the biosynthetic activity of a genetically-modified cell can be quantitatively measured and described as a percentage of the catalytic activity of an appropriate wild-type control. The catalytic activity exhibited by a genetically-modified polypeptide or the biosynthetic activity of a genetically-modified cell can be, for example, no more than 95%, no more than 90%, no more than 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, no more than 10%, no more than 5%, no more than 4%, no more than 3%, no more than 2%, no more than 1% of the activity, or 0% of the activity of a suitable wild-type control.

Alternatively, a decrease in catalytic activity can be expressed as a decrease in k_{cat} such as, for example, at least a two-fold decrease, at least a three-fold decrease, at least a four-fold decrease, at least a five-fold decrease, at least a six-fold decrease, at least a seven-fold decrease, at least an eight-fold decrease, at least a nine-fold decrease, at least a 10-fold decrease, at least a 15-fold decrease, or at least a 20-fold decrease in the k_{cat} value of the enzymatic conversion.

A decrease in catalytic activity also may be expressed in terms of an increase in Kₘ such as, for example, an increase in Kₘ of at least two-fold, at least three-fold, at least four-fold, at least five-fold, at least six-fold, at least seven-fold, at least an eight-fold, at least nine-fold, at least 10-fold, at least 15-fold, at least 20-fold, at least 25-fold, at least 30-fold, at least 35-fold, at least 40-fold, at least 45-fold, at least 50-fold, at least 75-fold, at least 100-fold, at least 150-fold, at least 200-fold, at least 230-fold, at least 250-fold, at least 300-fold, at least 350-fold, or at least 400-fold.

Thus, in another aspect, we describe herein methods for biosynthesis of pentanoic acid or 2-methylbutyric acid. Generally, the methods includes incubating a recombinant cell as described herein in medium that includes a carbon source under conditions effective for the recombinant cell to produce pentanoic acid or 2-methylbutyric acid. For producing pentanoic acid, the carbon source can include one or more of: glucose, pyruvate, L-aspartate, L-threonine, 2-ketobutyrate, 2-ketovalerate, 2-ketocaproate, or valeraldehyde. For producing 2-methylbutyric acid, the carbon source can include one or more of: glucose, pyruvate, L-aspartate, L-threonine, 2-ketobutyrate, 2-keto-3-methylvalerate, or 2-methyl butyraldehyde. In addition, the carbon sources for cell growth can be CO₂, cellulose, glucose, xylose, sucrose, arabinose, glycerol, etc. as long as the related carbon assimilation pathways are introduced in the engineered microbe.

In yet another aspect, we describe herein methods for introducing a heterologous polynucleotide into cell so that the host cell exhibits an increased ability to convert a carbon source to pentanoic acid or 2-methylbutyric acid. For cells to produce pentanoic acid, the heterologous polynucleotide can encode a polypeptide operably linked to a promoter so that the modified cell catalyzes conversion of the carbon source to pentanoic acid. In some of these embodiments, the carbon source can include one or more of glucose, pyruvate, L-aspartate, L-threonine, 2-ketobutyrate, 2-ketovalerate, 2-ketocaproate, or valeraldehyde. For cells to produce 2-methyl butyraldehyde, the heterologous polynucleotide can encode a polypeptide operably linked to a promoter so that the modified cell catalyzes conversion of the carbon source to 2-methyl butyraldehyde. In some of these embodiments, the carbon source can include one or more of glucose, pyruvate, L-aspartate, L-threonine, 2-ketobutyrate, 2-keto-3-methylvalerate, or 2-methyl butyraldehyde. The host cells for such methods can include, for example, any of the microbial species identified above with regard to the recombinant cells described herein.

As used in the preceding description, the term “and/or” means one or all of the listed elements or a combination of any two or more of the listed elements; the term “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiment can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Bacterial Strains, Reagents, Media and Cultivation

The E. coli strain used in this study was a threonine overproducer strain ATCC98082 which had threonine and homoserine exporter gene rhtA knocked out to ensure high intracellular level of threonine (Zhang et al., Proc Natl Acad Sci USA 2010; 107:6234-6239). The yqhD gene deletion strain was obtained from the Keio collection (Baba et al., Mol Syst Biol 2006; 2:2006.0008). It was transformed with plasmidpCP20 to remove the kanamycin resistance marker. This strain was transformed with plasmids pIPA1, pIPA2 and one of the pIPA4 to pIPA15 for production of 2-methylbutyric acid. For production of pentanoic acid, it was transformed with pIPA1, pIPA3 and any one of the pIPA4 to pIPA15.

XL1-Blue and XL10-Gold competent cells used for propagation of plasmids were from Stratagene (La Jolla, Calif.) while BL21 competent cells used for protein expression were from New England Biolabs (Ipswich, Mass.). All the restriction enzymes, QUICK LIGATION kit and PHUSION high-fidelity PCR kit were also from New England Biolabs.

A 2× YT rich medium (16 g/L Bacto-tryptone, 10 g/L yeast extract and 5 g/L NaCl) was used to culture the E. coli strains at 37° C. and 250 rpm. Antibiotics were added as needed (100 mg/L ampicillin, 25 mg/L kanamycin and 25 mg/L spectinomycin).

### Fermentation Procedure and HPLC Analysis

Fermentation experiments were carried out in triplicate and the data are presented as the mean values with error bars indicating the standard error. 250 μL of overnight cultures were transferred into 125 mL conical flasks containing 5 mL M9 medium supplemented with 5 g/L yeast extract, 40 g/L glucose, 10 mg/L thiamine, 100 mg/L ampicillin, 25 mg/L kanamycin and 25 mg/L spectinomycin. Protein expression was induced by adding 0.1 mM isopropyl-β-D-thiogalactoside (IPTG). 0.2 g CaCO₃ was added into the flask for neutralization of acids produced. After incubation for 48 hours at 30° C. and 250 rpm, samples were collected and analyzed using an Agilent 1260 Infinity HPLC containing a Aminex HPX 87H column (Bio-Rad Laboratories, Inc., Hercules, Calif.) equipped with a refractive-index detector. The mobile phase was 5 mM H₂SO₄ at a flow rate of 0.6 mL/minute. The column temperature was 35° C. and detection temperature was 50° C.

### Protein Expression and Purification

AldH was purified by cloning the gene into an expression plasmid encoding an N-terminal 6× His-tag to get pIPA16. This plasmid was then transformed into E. coli strain BL21. Cells were inoculated from an overnight pre-culture at 1/300 dilution and grown at 30° C. in 300 ml 2× YT rich medium containing 100 μg/L ampicillin. When the OD reached 0.6, IPTG was added to induce protein expression. Cell pellets were lysed by sonication in a buffer (pH 9.0) containing 250 mM NaCl, 2 mM DTT, 5 mM imidazole and 50 mM Tris. The enzyme was purified from crude cell lysate through Ni-NTA column chromatographyand buffer-exchanged using Amicon Ultra centrifugal filters (EMD Millipore Corp., Billerica, Mass.). Storage buffer (pH 8.0) containing 50 μM tris buffer, 1 mM MgSO₄ and 20% glycerol was used for AldH. The 100 μL of concentrated protein solutions were aliquoted into PCR tubes and flash frozen at −80° C. for long term storage. Protein concentration was determined by measuring UV absorbance at 280 nm. Purified KDH_{ba} and IPDC were available from an earlier study (Xiong et al., Sci Rep 2012; 2).

### Enzymatic Assay

Enzymatic assay of KDH_{ba} consisted of 0.5 mM NAD+ and valeraldehyde in the range of 50 μM to 400 μM in assay buffer (50 mM NaH₂PO₄, pH 8.0, 1 mM DTT) with a total volume of 78 μL. To start the reaction, 2 μL of 1 M KDH_{ba} was added and generation of NADH was monitored at 340 nm (extinction coefficient, 6.22 mM⁻¹ cm⁻¹). A similar protocol was used for AldH with 2-Methyl butyraldehyde concentrations in the range of 1 mM to 6 mM.

The activity of IPDC was measured using a coupled enzymatic assay method. Excess of an appropriate aldehyde dehydrogenase (AldH for 2-Keto-3-methylvalerate and KDH_{ba} for 2-Ketocaproate) was used to oxidize aldehyde into acid while cofactor NAD+ was reduced to NADH. The assay mixture contained 0.5 mM NAD+, 0.1 μM appropriate aldehyde dehydrogenase and corresponding 2-keto acid in the range of 1 mM to 8 mM in assay buffer (50 mM NaH₂PO₄, pH 6.8, 1 mM MgSO4, 0.5 mM ThDP) with a total volume of 78 μL. To start the reaction, 2 μL of 1 μM IPDC was added and generation of NADH was monitored at 340 nm. Kinetic parameters (k_{cat} and K_{M}) were determined by fitting initial rate data to the Michaelis-Menten equation.

TABLE 2
Strains and primers used in the study

| Strain or | | Reference |
|---|---|---|
| plasmid | Description | or source |
| Strains | | |
| PA1 | ATCC98082(ΔrhtA, ΔyqhD) | This study |
| XL10- | Tet^{r}Δ (mcrA)183 Δ (mcrCB-hsdSMR-mrr)173 | Stratagene |
| Gold | endA1 supE44 thi-1 recA1 gyrA96 relA1 lac Hte | |
| | [F′ proAB lacI^{q}ZDM15 Tn10 (Tet^{r}) Amy Cam^{r}] | |
| XL1- | recA1 endA1 gyrA96 thi-1 hsdR17 supE44 relA1 | Stratagene |
| Blue | lac [F′ proAB lacI^{q}ZΔM15 Tn10 (Tet^{r})] | |
| Plasmids | | |
| pIPA1 | psc101 ori; Spec^{r}; P_{L}lacO1: thrA-thrB-thrC | a |
| pIPA2 | p15A ori; Kan^{r}; P_{L}lacO1: ilvA-ilvG-ilvM- | This study |
| | ilvC-ilvD | |
| pIPA3 | p15A ori; Kan^{r}; P_{L}lacO1: ilvA-leuA-leuB- | This study |
| | leuC-leuD | |
| pIPA4 | ColE1 ori; Amp^{r}; P_{L}lacO1: kivD-padA | b |
| pIPA5 | ColE1 ori; Amp^{r}; P_{L}lacO1: kivD-aldB | b |
| pIPA6 | ColE1 ori; Amp^{r}; P_{L}lacO1: kivD-gabD | b |
| pIPA7 | ColE1 ori; Amp^{r}; P_{L}lacO1: kivD-KDH_{ba} | b |
| pIPA8 | ColE1 ori; Amp^{r}; P_{L}lacO1: kivD-aldH | b |
| pIPA9 | ColE1 ori; Amp^{r}; P_{L}lacO1: kivD-ydcW | b |
| pIPA10 | ColE1 ori; Amp^{r}; P_{L}lacO1: kivD V461A/F381L- | c |
| | padA | |
| pIPA11 | ColE1 ori; Amp^{r}; P_{L}lacO1: kivD V461A/F542L- | c |
| | padA | |
| pIPA12 | ColE1 ori; Amp^{r}; P_{L}lacO1: kivD V461A/M538A- | c |
| | padA | |
| pIPA13 | ColE1 ori; Amp^{r}; P_{L}lacO1: IPDC-padA | c |
| pIPA14 | ColE1 ori; Amp^{r}; P_{L}lacO1: IPDC-aldH | This study |
| pIPA15 | ColE1 ori; Amp^{r}; P_{L}lacO1: IPDC-KDH_{ba} | c |
| pIPA16 | ColE1 ori; Amp^{r}; P_{L}lacO1: 6xhis-aldH | This study |

a. Zhang et al., Proc Natl Acad Sci USA 2008; 105: 20653-20658.
b. Zhang et al., ChemSusChem 2011; 4: 1068-1070.
c. Xiong et al., Sci Rep 2012; 2.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

Sequence Listing Free Text

SeqID: YP_004731039.1 GI:340000155
Protein name: putative decarboxylase [Salmonella bongori NCTC 12419]
SEQ ID NO: 1

| | |
|---|---|
| 1 | mqtpytvady lldrlagcgi dhlfgvpgdy nlqfldhvid hptlrwvgca nelnaayaad |
| 61 | gyarmsgaga llttfgvgel saingiagsy aeyvpvlhiv gapcsdaqqr gelmhhtlgd |
| 121 | gdfrhfyrms qaisaasavl neqnacyeid rvlgemltah rpcyillpad vakkpaippt |
| 181 | etlmlpanka qssvetafry harqclmnsr rialladfla rrfglrpllq rwmvetpiah |
| 241 | atllmgkglf neqhpnfvgt ysagasskev rgaiedadmv icvgtrfvdt ltagftqqlp |
| 301 | aertleiqpy asrigdswft lpmelaysil relclecafa psptrssgqs ipvekgaltq |
| 361 | enfwqtlqqf ikpgdiilvd qgtaafgaaa lslpdgaevl vqplwgsigy slpaafgaqt |
| 421 | acpdrrvili igdgaaqlti qemgsmlrde qapiilllnn egytveraih gaaqryndia |
| 481 | swnwtqipqa lsaaqqaecw rvtgaiglee ilarlarpqr lslievmlpk adlpellrtv |
| 541 | tralemrngg |

SeqID: ZP_03365331.1 GI:213583505
Protein name: putative decarboxylase [Salmonella enterica subsp. enterica
serovar Typhi str. E98-0664]
SEQ ID NO: 2

| | |
|---|---|
| 1 | ldhvidhptl rwvgcaneln aaytadgyar msgagalltt fgvgelsain giagsyaeyv |
| 61 | pvlhivgapc saaqqrgelm hhtlgdgdfr hfyrmsqais aasaildeqn acfeidrvlg |
| 121 | emlaarrpgy imlpadvakk taippteala lpvheaqsgv etafryharq clmnsrrial |
| 181 | ladflagrfg lrpllqrwma etpiahatll mgkglfdeqh pnfvgtysag asskevrqai |
| 241 | edadrvicvg trfvdtltag ftqqlpaert leiqpyasri getwfnlpma qaystlrelc |
| 301 | lecafapppt rsagqpvrid kgeltqesfw qtlqqclkpg diilvdqgta afgaaalslp |
| 361 | dgaevvvqpl wgsigyslpa afgaqtacpd rrviliigdg aagltiqemg smlrdgqapv |
| 421 | illlnndgyt veraihgaaq ryndiaswnw tqippalnaa qqaecwrvtq aiglaevler |
| 481 | larpqrlsfi evmlpkadlp ellrtvtral earngg |

SeqID: YP_001569550.1 GI:161502438
Protein name: hypothetical protein SARI_00479 [Salmonella enterica subsp.
arizonae serovar 62:z4,z23:-- str. RSK2980]
SEQ ID NO: 3

| | |
|---|---|
| 1 | mqtpytvady lldrlagcgi ghlfgvpgdy nlqfldhvid hptlrwvgca nelnaayaad |
| 61 | gyarmsgaga llttfgvgel saingiagsy aeyvpvlhiv gapcsaaqqr gelmhhtlgd |
| 121 | gdfhhfyrms gaisagsail negnacfeid rvlgemvaar rpgyimlpad vakktaippi |
| 181 | ealtlpahet qngvetafry rarqclmnsr rialladfla rrfglrpllq rwmaetsiah |
| 241 | atllmgkglf deqhpnfvgt ysagasskav rgaiedadmv icvgtrfvdt ltagftqqlp |
| 301 | aertleiqpy asrigetwfn lpmagaystl relclecafa ppptrpvcqp vgiekgeltq |
| 361 | enfwqtlqqy lkpgdiilvd qgtaafgaaa lslpdgaevv vqplwgsigy slpaafgaqt |
| 421 | acpdrrvili igdgaaqlti qemgsmlrdg qapiilllnn dgytveraih gaagryndia |
| 481 | swnwtqipqa lnaaqqaecw rvtgaiglae vlerlarpqr lsfievmlpk adlpellrtv |
| 541 | tralearngg |

SeqID: YP_149772.1 GI:56412697
Protein name: decarboxylase [Salmonella enterica subsp. enterica serovar
Paratyphi A str. ATCC 9150]
SEQ ID NO: 4

| | |
|---|---|
| 1 | mqnpytvady lldrlagcgi ghlfgvpgdy nlqfldhvid hptlrwvgca nelnaayaad |
| 61 | gyarmsgaga llttfgvgel saingiagsy aeyvpvlhiv gapcsaaqqr gelmhhtlgd |
| 121 | gdfrhfyrms qaisaasail deqnacfeid rvlgemlaar rpgyimlpad vakktaippt |
| 181 | qaltlpvhea qsgvetafry harqclmnsr rialladfla grfglrpllq rwmaetpiah |
| 241 | atllmekglf deqhpnfvgt ysagasskev rgaiedadry icvgtrfvdt ltagftqqlp |
| 301 | aertleiqpy asrigetwfn lpmagaystl relclecafa ppptrsagqp vridkgeltq |
| 361 | esfwqtlqqy lkpgdivlvd qgtaafgaaa lslpdgaevv vgplwgsigy slpaafgaqt |
| 421 | acpdrrvili igdgaaqlti qemgsmlrdg qapvilllnn ggytveraih gaaqryndia |
| 481 | swnwtqippa lnaaqqvecw rvagaiglae vlerlarpqr lsfievmlpk adlpellrtv |
| 541 | tralearngg |

SeqID: ZP_02654846.1 GI:168229788
Protein name: indole-3-pyruvate decarboxylase [Salmonella enterica subsp.
enterica serovar Kentucky str. CDC 191]
SEQ ID NO: 5

| | |
|---|---|
| 1 | mqnpytvady lldrlagcgi ghlfgvpgdy nlqfldhvid hptlrwvgca nelnaayaad |
| 61 | gyarmsgaga llttfgvgel saingiagsy aeyvpvlhiv gapcsaaqqr gelmhhtlgd |
| 121 | gdfrhfyrms qaisvasail deqnacfeid rvlgemfaar rpgyimlpad vakktaippt |
| 181 | qaltlpvhea gsgvetafry harqclmnsr rialladfla grfglrpllq rwmvetpiah |
| 241 | atllmgkglf deqhpnfvgt ysagasskev rgaiedadry icvgtrfvdt ltagftqqlp |
| 301 | aertleiqpy asrigetwfn lpmagaystl relclecafa ppptrsagqp vridkgeltq |
| 361 | esfwqtlqqy lkpgdivlvd qgtaafgaaa lslpdgaevv vqplwgsigy slpaafgaqt |
| 421 | acpdrrvili igdgaaqlti qemgsmlrdg qapvilllnn dgytveraih gaaqryndia |
| 481 | swnwtqippa lnaaqqaecw rvtgaiglae vlerlvrpqr lsfievmlpk adlpellrtv |
| 541 | tralearngg |

SeqID: ZP_03220347.1 GI:204929204
Protein name: indole-3-pyruvate decarboxylase [Salmonella enterica subsp.
enterica serovar Javiana str. GA_MM04042433]
SEQ ID NO: 6

| | |
|---|---|
| 1 | mqnpytvady lldrlagcgi ghlfgvpgdy nlqfldhvid hptlrwvgca nelnaayaad |
| 61 | gyarmsgaga llttfgvgel saingiagsy aeyvpvlhiv gapcsaaqqr gelmhhtlgd |
| 121 | gdfrhfyrms qaisvasail yeqnacfeid rvlgemlaar rpgyimlpad vakktaippt |
| 181 | ealalpvhea qsgvetafry harqclmnsr rialladfla grfglrpllq rwmaetpiah |
| 241 | atllmgkglf deqhpnfvgt ysagasskev rgaiedadry icvgtrfvdt ltagftqqlp |
| 301 | aertleiqpy asrigetwfn lpmagaystl relclecafa ppptrstgqp vridkgeltq |
| 361 | esfwqtlqqy lkpgdiilvd qgtaafgaaa lslpdgaevv vqplwgsigy slpaafgaqt |
| 421 | acpdrrvili igdgaaglti gemgsmlrdg qapvilllnn dgytveraih gaaqryndia |
| 481 | swnwtqippa lnaaqqaecw rvtgaiglae vlerlarpqr lsfievmlpk adlpellrtv |
| 541 | tralearng |

SegED:NP_456948.1 C116761331
Protein name: decarboxylase [Salmonella enterica subsp. enterica serovar
Typhi str. CT18]
SEQ ID NO: 7

| | |
|---|---|
| 1 | mqnpytvady lldrlagcgi ghlfgvpgdy nlqfldhvid hptlrwvgca nelnaaytad |
| 61 | gyarmsgaga llttfgvgel saingiagsy aeyvpvlhiv gapcsaaqqr gelmhhtlgd |
| 121 | gdfrhfyrms qaisaasail deqnacfeid rvlgemlaar rpgyimlpad vakktaippt |
| 181 | ealalpvhea qsgvetafry harqclmnsr rialladfla grfglrpllq rwmaetpiah |
| 241 | atllmgkglf deqhpnfvgt ysagasskev rqaiedadry icvgtrfvdt ltagftqqlp |
| 301 | aertleiqpy asrigetwfn lpmagaystl relclecafa ppptrsagqp vridkgeltq |
| 361 | esfwqtlqqc lkpgdiilvd qgtaafgaaa lslpdgaevv vqplwgsigy slpaafgaqt |
| 421 | acpdrrvili igdgaaqlti qemgsmlrdg qapvilllnn dgytveraih gaaqryndia |
| 481 | swnwtqippa lnaaqqaecw rvtqaiqlae vlerlarpqr lsfievmlpk adlpellrtv |
| 541 | tralearngg |

SeqID: ZP_03215433.1 GI:200388821
Protein name: indole-3-pyruvate decarboxylase [Salmonella enterica subsp.
enterica serovar Virchow str. SL491]
SEQ ID NO: 8

| | |
|---|---|
| 1 | mqnpytvady lldrlagcgi ghlfgvpgdy nlqfldhvid hptlrwvgca nelnaayaad |
| 61 | gyarmsgaga llttfgvgel saingiagsy aeyvpvlhiv gapcsaaqqr gelmhhtlgd |
| 121 | gdfrhfyrms qaisvasail deqnacfeid rvlgemlvar rpgyimlpad vakktaippt |
| 181 | qalalpvhea qsgvetafry harqclmnsr rialladfla grfglrpllq rwmaetpiah |
| 241 | atllmgkglf deqhpnfvgt ysagasskev rqaiedadry icvgtrfvdt ltvgftqqlp |
| 301 | tertleiqpy asrigetwfn lpmagaystl relclecafa ppptrsagqp vridkgeltq |
| 361 | esfwqtlqqy lkpgdiilvd qgtaafgaaa lslpdgakvv vqplwgsigy slpaafgaqt |
| 421 | acpdrrvili igdgaaqlti qemgsmlrdg qapvilllnn dgytveraih gaaqryndia |
| 481 | swnwtqippa lnaaqqaecw rvtgaiglae vlerlarpqr lsfievmlpk adlpellrtv |
| 541 | tralearngg |

SeqID: EFY11092.1 GI:322614157
Protein name: indole-3-pyruvate decarboxylase [Salmonella enterica subsp.
enterica serovar Montevideo str. 315996572]
SEQ ID NO: 9

| | |
|---|---|
| 1 | mqnpytvady lldrlagcgi ghlfgvpgdy nlqfldhvid hptlrwvgca nelnaayaad |
| 61 | gyarmsgtga llttfgvgel saingiagsy aeyvpvlhiv gapcsaaqqr gelmhhtlgd |
| 121 | gdfrhfyrms qaisvassil deqnacfeid rvlgemlaar rpgyimlpad vakktaippt |
| 181 | ealalpvhea qsgvetafry harqclmnsr rialladfla grfglrpllq rwmaetpiah |
| 241 | atllmgkglf deqhpnfvgt ysagasskev rqaiedadry icvgtrfvdt ltagftqqlp |
| 301 | aertleiqpy asrigetwfn lpmagaystl relclecafa ppptrsagqp vridkgeltq |
| 361 | esfwqtlqqy lkpgdiilvd qgtaafgaaa lslpdgaevv vqplwgsigy slpaafgaqt |
| 421 | acpdrrvili igdgaaqlti qemgsmlrdg qapvilllnn dgytveraih gaaqryndia |
| 481 | swnwtqippa lnaaqqaecw rvtgaiglae vlerlarpqr lsfievmlpk adlpellrtv |
| 541 | tralearngg |

SeqID: ZP_02662493.1 GI:168237435
Protein name: indole-3-pyruvate decarboxylase (Indolepyruvatedecarboxylase)
[Salmonella enterica subsp. enterica serovar Schwarzengrund str. SL480]
SEQ ID NO: 10

| | |
|---|---|
| 1 | mqnpytvady lldrlagcgi ghlfgvpgdy nlqfldhvid hptlrwvgca nelnaayaad |
| 61 | gyarmsgtga llttfgvgel saingiagsy aeyvpvlhiv gapcsaaqqr gelmhhtlgd |
| 121 | gdfrhfyrms qaisvasail deqnacfeid rvlgemlaar rpgyimlpad vakktaippt |
| 181 | ealalpvhea qsgvetafry harqclmnsr rialladfla grfglrpllq rwmaetpiah |
| 241 | atllmgkglf deqhpnfvgt ysagasskev rqaiedadry icvgtrfvdt ltagftqqlp |
| 301 | aertleiqpy asrigetwfn lpmagaystl relclecafa ppptrsagqp vridkgeltq |
| 361 | esfwqtlqqy lkpgdiilvd qgtaafgaaa lslpdgaevv vqplwgsigy slpaafgaqt |
| 421 | acpdrrvilv igdgaaqlti qemgsmlrdg qapvilllnn dgytveraih gaaqryndia |
| 481 | swnwtqippa lnaaqqaecw rvtgaiglae vlerlarpqr lsfievmlpk adlpellrtv |
| 541 | tralearngg |

SeqID: YP_217395.1 GI:62180978
Protein name: putative thiamine pyrophosphate enzyme [Salmonella enterica
subsp. enterica serovar Choleraesuis str. SC-B67]
SEQ ID NO: 11

| | |
|---|---|
| 1 | mqnpytvady lldrlagcgi ghlfgvpgdy nlqfldhvid hptlrwvgca nelnaayaad |
| 61 | gyarmsgaga llttfgvgel saingiagsy aeyvpvlhiv gapcsaaqqr gelmhhtlgd |
| 121 | gdfrhfyrms qaisaasail deqnacfeid rvlgemlaar rpgyimlpad vakktaippt |
| 181 | qalalpvhea qsgvetafry harqclmnsr rialladfla grfglrpllq rwmaetpiah |
| 241 | atllmgkglf deqhpnfvgt ysagasskev rqaiedadry icvgtrfvdt ltagftqqlp |
| 301 | tertleiqpy alrigetwfn lpmagaystl relclecafa ppptrsagqp vridkgeltq |
| 361 | esfwqtlqqy lkpgdiilvd qgtaafgaaa lslpdgaevv vqplwgsigy slpaafgaqt |
| 421 | acpdrrvili igdgaaqlti qemgsmlrdg qapvilllnn dgytveraih gaaqryndia |
| 481 | swnwtqippa lnaaqqaecw rvtgaiglae vlerlarpqr lsfievmlpk aelpellrtv |
| 541 | tralearngg |

SeqID: ZP_02829849.1 GI:168817849
Protein name: indole-3-pyruvate decarboxylase [Salmonella enterica subsp.
enterica serovar Weltevreden str. HI_N05-537]
SEQ ID NO: 12

| | |
|---|---|
| 1 | mqnpytvady lldrlagcgi ghlfgvpgdy nlqfldhvid hptlrwvgca nelnaaytad |
| 61 | gyarmsgaga llttfgvgel saingiagsy aeyvpvlhiv gapcsaaqqr gelmhhtlgd |
| 121 | gdfrhfyrms qaisvasail deqnacfeid rvlgemlaar rpgyimlpad vakktaippt |
| 181 | qalalpvhea qsgvetafry harqclmnsr rialladfla grfglrpllq rwmaetpiah |
| 241 | atllmgkglf deqhpnfvgt ysagasskev rqaiedadry icvgtrfvdt ltagftqqlp |
| 301 | aertleiqpy asrigetwfn lpmagaystl relclecafa ppptrsagqp vridkgeltq |
| 361 | esfwqtlqqy lkpgdiilvd qgtaafgaaa lslpdgaevv vqplwgsigy slpaafgaqt |
| 421 | acpdrrvili igdgaaqlti qemgsmlrdg qapvilllnn dgytveraih gaaqryndia |
| 481 | swnwtqippa lnaaqqaecw rvtqgiqlae vlerlarpqr lsfievmlpk adlpellrtv |
| 541 | tralearngg |

SeqtD:ZP_02683535.1 C4:168261562
Protein name: indole-3-pyruvate decarboxylase [Salmonella enterica subsp.
enterica serovar Hadar str. RI_05P066]
SEQ ID NO: 13

| | |
|---|---|
| 1 | mqnpytvady lldrlagcgi ghlfgvpgdy nlqfldhvid hptlrwvgca nelnaayaad |
| 61 | gyarmsgaga llttfgvgel saingiagsy aeyvpvlhiv gapcsaaqqr gelmhhtlgd |
| 121 | gdfrhfyrms qaisaasail deqnacfeid rvlgemlaar rpgyimlpad vakktaippt |
| 181 | qalalpvhea qsgvetafry harqclmnsr rialladfla grfglrpllq rwmaetpiah |
| 241 | atllmgkglf deqhpnfvgt ysagasskev rgaiedadry icvgtrfvdt ltagftqqlp |
| 301 | tertleiqpy asrigetwfn lpmagaystl relclecafa ppptrsagqp vridkgeltq |
| 361 | esfwqtlqqy lkpgdiilvd qgtaafgaaa lslpdgakvv vqplwgsigy slpaafgaqt |
| 421 | acpdrrvili igdgaaqlti qemgsmlrdg qapvilllnn dgytveraih gaaqryndia |
| 481 | swnwtqippa lnaaqqaecw rvtgaiglae vlerlarpqr lsfievmlpk adlpellrtv |
| 541 | tralearngg |

SeqID: YP_002227320.1 GI:205353519
Protein name: decarboxylase [Salmonella enterica subsp. enterica serovar
Gallinarum str. 287/91]
SEQ ID NO: 14

| | |
|---|---|
| 1 | mqnpytvady lldrlagcgi ghlfgvpgdy nlqfldhvid hptlrwvgca nelnaayaad |
| 61 | gyarmsgaga llttfgvgel saingiagsy aeyvpvlhiv gapcsaaqqr gelmhhtlgd |
| 121 | gdfrhfyrms qaisaasail deqnacfeid rvlgemlaar rpgyimlpad vakktaippt |
| 181 | qalalpvhea qsgvetafry hargclmnsr rialladfla grfglrpllq rwmaetpiah |
| 241 | atllmgkglf deqhpnfvgt ysagasskev rgaiedadry icvgtrfvdt ltagftqqlp |
| 301 | tertleiqpy asrigetwfn lpmagaystl relclecafa ppptrsagqp vridkgeltq |
| 361 | esfwqtlqqy lkpgdiilvd qgtaafgaaa lslpdgaevv vqplwgsigy slpaafgaqt |
| 421 | acpdrrvili igdgaaqlti qemgsmlrdg qapvilllnn dgytveraih gaarryndia |
| 481 | swnwtqippa lnaaqqaecw rvtgaiglae vlerlarpqr lsfievmlpk adlpellrtv |
| 541 | tralearngg |

SeqID: YP_002636855.1 GI:224583057
Protein name: decarboxylase [Salmonella enterica subsp. enterica serovar
Paratyphi C strain RKS4594]
SEQ ID NO: 15

| | |
|---|---|
| 1 | mqnpytvady lldrlagcgi ghlfgvpgdy nlqfldhvid hptlrwvgca nelnaayaad |
| 61 | gyarmsgaga llttfgvgel saingiagsy aeyvpvlhiv gapcsaaqqr gelmhhtlgd |
| 121 | gdfrhfyrms gaisaasail deqnacfeid rvlgemlaar rpgyimlpad vakktaippt |
| 181 | qalalpvhea qsgvetafry hargclmnsr rialladfla grfglrpllq rwmaetpiah |
| 241 | atllmgkglf deqhpnfvgt ysagasskev rgaiedadry icvgtrfvdt ltagftqqlp |
| 301 | tertleiqpy asrigetwfn lpmagaystl relclecafa ppptrsagqp vridkgeltq |
| 361 | esfwqtlqqy lkpgdiilvd qgtaafgaaa lslpdgaevv vqplwgaigy slpaafgaqt |
| 421 | acpdrrvili igdgaaqlti qemgsmlrdg qapvilllnn dgytveraih gaagryndia |
| 481 | swnwtqippa lnaaqqaecw rvtgaiglae vlerlarpqr lsfievmlpk adlpellrtv |
| 541 | tralearngg |

SeqED:ZP_04656662.1 GI:238912825
Protein name: indole-3-pyruvate decarboxylase [Salmonella enterica subsp.
enterica serovar Tennessee str. CDC07-0191]
SEQ ID NO: 16

| | |
|---|---|
| 1 | mqnpytvady lldrlagcgi ghlfgvpgdy nlqfldhvid hptlrwvgca nelnaayaad |
| 61 | gyarmsgaga llttfgvgel saingiagsy aeyvpvlhiv gapcsaaqqr gelmhhtlgd |
| 121 | gdfrhfyrms qaisaasail degnacfeid rvlgemfaar rpgyimlpad vakktaippt |
| 181 | qaltlpvhea qsgvetafry harqclmnsr rialladfla grfglrpllq rwmaetpiah |
| 241 | atllmgkglf deqhpnfvgt ysagasskev rgaiedadry icvgtrfvdt ltagftqqlp |
| 301 | aertleiqpy asrigetwfn lpmagaystl relclecafa ppptrsagqp vridkgeltq |
| 361 | esfwqtlqqy lkpgdivlvd qgtaafgaaa lslpdgaevv vqplwgsigy slpaafgaqt |
| 421 | acpdrrvili igdgaaqlti qemgsmlrdg qapvilllnn dgytveraih gaaqryndia |
| 481 | swnwtqippa lnaaqqaecw rvtgaiglae vlerlarpqr lsfievmlpk adlpellrtv |
| 541 | tralearngg |

SeqID: CBW18475.1 GI:301158962
Protein name: putative decarboxylase [Salmonella enterica subsp. enterica
serovar Typhimurium str. SL1344]
SEQ ID NO: 17

| | |
|---|---|
| 1 | mqnpytvady lldrlagcgi ghlfgvpgdy nlqfldhvid hptlrwvgca nelnaayaad |
| 61 | gyarmsgaga llttfgvgel saingiagsy aeyvpvlhiv gapcsaaqqr gelmhhtlgd |
| 121 | gdfrhfyrms qaisaasail degnacfeid rvlgemlaar rpgyimlpad vakktaippt |
| 181 | galalpvhea qsgvetafry harqclmnsr rialladfla grfglrpllq rwmaetpiah |
| 241 | atllmgkglf deqhpnfvgt ysagasskev rgaiedadry icvgtrfvdt ltarftqqlp |
| 301 | aertleiqpy asrigetwfn lpmagaystl relclecafa ppptrsagqp vridkgeltq |
| 361 | esfwqtlqqy lkpgdiilvd qgtaafgaaa lslpdgaevv lqplwgsigy slpaafgaqt |
| 421 | acpdrrvili igdgaaqlti qemgsmlrdg gapvilllnn dgytveraih gaagryndia |
| 481 | swnwtqippa lnaaqqaecw rvtgaiglae vlerlarpqr lsfievmlpk adlpellrtv |
| 541 | tralearngg |

SeqID: YP_002147363.1 GI:197247765
Protein name: indole-3-pyruvate decarboxylase [Salmonella enterica subsp.
enterica serovar Agona str. SL483]
SEQ ID NO: 18

| | |
|---|---|
| 1 | mqnpytvady lldrlagcgi ghlfgvpgdy nlqfldhvid hptlrwvgca nelnaayaad |
| 61 | gyarmsgaga llttfgvgel saingiagsy aeyvpvlhiv gapcsaaqqr gelmhhtlgd |
| 121 | gdfrhfyrms qaisaasail degnacfeid rvlgemlaar rpgyimlpad vakktaippt |
| 181 | qalalpvhea qsgvetafry hargclmnsr rialladfla grfglrpllq rwmaetpiah |
| 241 | atllmgkglf deqhpnfvgt ysagasskev rgaiedadry icvgtrfvdt ltagftqqlp |
| 301 | aertleiqpy asrigetwfn lpmagaystl relclecafa ppptrsagqp vridkgeltq |
| 361 | esfwqtlqqy lkpgdivlvd qgtaafgaaa lslpdgaevv vqplwgsigy slpaafgaqt |
| 421 | acpdrrvili igdgaaqlti qemgsmlrdg qapvilllnn dgytveraih gaagryndia |
| 481 | swnwtqippa lnaaqqaecw rvtgaiglae vlerlarpqr lsfievmlpk adlpellrtv |
| 541 | tralearngg |

SeqID: ZP_02667483.1 GI:168242551
Protein name: indole-3-pyruvate decarboxylase [Salmonella enterica subsp.
enterica serovar Heidelberg str. SL486]
SEQ ID NO: 19

| | |
|---|---|
| 1 | mqnpytvady lldrlagcgi ghlfgvpgdy nlqfldhvid hptlrwvgca nelnaayaad |
| 61 | gyarmsgaga llttfgvgel saingiagsy aeyvpvlhiv gapcsaaqqr gelmhhtlgd |
| 121 | gdfrhfyrms qaisaasail deqnacfeid rvlgemlaar rpgyimlpad vakktaippt |
| 181 | qalalpvhea qsgvetafry harqclmnsr rialladfla grfglrpllq rwmaetpiah |
| 241 | atllmgkglf deqhpnfvgt ysagasskev rgaiedadry icvgtrfvdt ltagftqqlp |
| 301 | tertleiqpy asrigetwfn lpmagaystl relclecafa ppptrsagqp vridkgeltq |
| 361 | esfwqtlqqy lkpgdiilvd qgtaafgaaa lslpdgaevv vqplwgsigy slpaafgaqt |
| 421 | acpdrrvili igdgaaqlti qemgsmlrdg qapvilllnn dgytveraih gaaqryndia |
| 481 | swnwtqippa lnaaqqaecw rvtgaiglae vlerlarpqr lsfievmlpk adlpellrtv |
| 541 | tralearngg |

SeqID: YP_001586815.1 GI:161612850
Protein name: hypothetical protein SPAB_00555 [Salmonella enterica subsp.
enterica serovar Paratyphi B str. SPB7]
SEQ ID NO: 20

| | |
|---|---|
| 1 | mqnpytvady lldrlagcgi ghlfgvpgdy nlqfldhvid hptlrwvgca nelnaayaad |
| 61 | gyarmsgaga llttfgvgel saingiagsy aeyvpvlhiv gapcsaaqqr gelmhhtlgd |
| 121 | gdfrhfyrms qaisaasail deqnacfeid rvlgemlaar rpgyimlpad vakktaippt |
| 181 | qalalpvhea qsgvetafry hargclmnsr rialladfla grfglrpllq rwmaetpiah |
| 241 | atllmgkglf deqhpnfvgt ysagasskev rgaiedadry icvgtrfvdt ltagftqqlp |
| 301 | aertleiqpy asrigetwfn lpmagaystl relclecafa ppptrsagqp vridkgeltq |
| 361 | esfwqtlqqy lkpgdiilvd qgtaafgaaa lslpdgaevv vqplwgsigy slpaafgaqt |
| 421 | acpdrrvili igdgaaqlti qemgsmlrdg qapvilllnn dgytveraih gaagryndia |
| 481 | swnwtqippa lnaaqqaecw rvtgaiglae vlerlarpqr lsfievmlpk adlpellrtv |
| 541 | tralearngg |

SeqID: NP_461346.1 GI:16765731
Protein name: indolepyruvate decarboxylase [Salmonella enterica subsp.
enterica serovar Typhimurium str. LT2]
SEQ ID NO: 21

| | |
|---|---|
| 1 | mqnpytvady lldrlagcgi ghlfgvpgdy nlqfldhvid hptlrwvgca nelnaayaad |
| 61 | gyarmsgaga llttfgvgel saingiagsy aeyvpvlhiv gapcsaaqqr gelmhhtlgd |
| 121 | gdfrhfyrms qaisaasail deqnacfeid rvlgemlaar rpgyimlpad vakktaippt |
| 181 | qalalpvhea qsgvetafry harqclmnsr rialladfla grfglrpllq rwmaetpiah |
| 241 | atllmgkglf deqhpnfvgt ysagasskev rgaiedadry icvgtrfvdt ltagftqqlp |
| 301 | aertleiqpy asrigetwfn lpmagaystl relclecafa ppptrsagqp vridkgeltq |
| 361 | esfwqtlqqy lkpgdiilvd qgtaafgaaa lslpdgaevv lqplwgsigy slpaafgaqt |
| 421 | acpdrrvili igdgaaqlti qemgsmlrdg qapvilllnn dgytveraih gaagryndia |
| 481 | swnwtqippa lnaaqqaecw rvtgaiglae vlerlarpqr lsfievmlpk adlpellrtv |
| 541 | tralearngg |

SeqID: YP_004156811.1 GI:319795171
Protein name: aldehyde dehydrogenase [Variovorax paradoxus EPS]
SEQ ID NO: 22

| | |
|---|---|
| 1 | mtatytdtrl lidnewvdat ggktldvvnp atgkvigkva hasiadldra laaaqrgfdk |
| 61 | wrntpanera avmrraagli reragdiakl ltqeqgkpla eakgetlaaa diiewfadeg |
| 121 | rrvygrivps rnlaaqqlvl keplgpvaaf tpwnfpinqi vrklgaalat gcsflvkape |
| 181 | etpaspaall qafvdagipp gtvglvfgnp aeisnyliah piirkvtftg stpvgkqlaa |
| 241 | lagshmkrvt melgghapvi vaedadvala vkaagaakfr nagqvcispt rflvhnslre |
| 301 | efartivkyt eglklgdgla egttigplan arrltamayv ledarkkgat vaaggervgd |
| 361 | sgnffaptvl tdvpldadvf nnepfgpiaa irgfdtleea iaeanrlpfg lagyaftksi |
| 421 | knahllsqkl elgmlwinqp atpspempfg gvkdsgygse ggpealeayl ntkaysilgv |

SeqID: YP_002945800.1 GI:239816890
Protein name: aldehyde dehydrogenase (NAD(+)) [Variovorax paradoxus S110]
SEQ ID NO: 23

| | |
|---|---|
| 1 | mtatytdtrl lidnewvdat ggktldvvnp atgkaigkva hasiadldra laaaqrgfek |
| 61 | wrntpanera avmrraagli rerapeiakl ltqeqgkpla eakgetlaaa diiewfadeg |
| 121 | rrvygrivps rnlaaqqlvi keplgpvaaf tpwnfpinqi vrklgaalat gcsflvkape |
| 181 | etpaspaall qafvdagipp gtvglvfgnp aeisnylish piirkvtftg stpvgkqlaa |
| 241 | lagshmkrvt melgghapvi vaedadvala vkaagaakfr nagqvcispt rflvhnslre |
| 301 | efartivkyt eglklgdgla egttlgplan arrltamahv lddarkkgat vaaggervgd |
| 361 | tgnffaptvl tdvpldadvf nnepfgpiaa irgfdtleea iaeanrlpfg lagyaftrsi |
| 421 | knahllsqkl elgmlwinqp aapspempfg gvkdsgygse ggpealeayl ntkaysimsv |

SeqID: ZP_03268788.1 GI:209520010
Protein name: Aldehyde Dehydrogenase [Burkholderia sp. H160]
SEQ ID NO: 24

| | |
|---|---|
| 1 | maissytdtr llingewcda vsgktldvin patggaigkv ahagiadldr aldaaqrgfe |
| 61 | awrkvpaher atimrkaaal vreraadigr lmtqeqgkpf aearvevlaa adiiewfade |
| 121 | grrvygrivp srnlaahsqv lkepigpvaa ftpwnfpvnq vvrklsasla cgcsflvkap |
| 181 | eetpaspaal lqafveagvp pgtvglvfgd paeissylip hpvirkvtft gstpvgkqla |
| 241 | alagthmkra tmelgghapv ivaedadval avkaagaakf rnagqvcisp trflvhnsir |
| 301 | eefaaalvkh aeslklgdgl aegttlgpla narrlsamak vvedarktga kvatggervg |
| 361 | segnffaatv ltdvpleadv fnnepfgpva airgfdtlde aiteanrlpy glagyaytks |
| 421 | fanvhqlsqr mevgmlwinq patptpempf ggvkdsgygs eggpeameay lvtkavtima |
| 481 | v |

SeqID: YP_004022361.1 GI:312602516
Protein name: 6-oxohexanoate dehydrogenase [Burkholderia rhizoxinica HKI
454]
SEQ ID NO: 25

| | |
|---|---|
| 1 | mvtssytdtr llidgqwcda asgktldvvn patgqvigry ahagiadldr alaaaqrgfd |
| 61 | twrkvpvher aatmrkaatl vreraegiar lmtqeqgkpf aearievlsa adiiewfade |
| 121 | grrvygrivp srnlavqqsv lkepigpvaa ftpwnfpvnq vvrklsaala cgcsflvkap |
| 181 | eetpaspagl lqafvdagvp agtiglvfgd paaissylia hpvirkvtft gstpvgkqla |
| 241 | alagahmkra tmelgghapv ivaedadial aikaaggakf rnagqvcisp trflvhnsir |
| 301 | eafeaalvkh acolklgdgl aqgttlgpla narrltamtr ivenaratga tvatggervg |
| 361 | sagnffaptv ltnvprdadv fnqepfgpva avrgfdrled aiaeanrlpy glagyaftrs |
| 421 | vrnvhllshq levgmlwinq patpwpempf ggvkdsgygs eggpeameay lvtkaysvaa |
| 481 | v |

SeqID: YP_003605215.1 GI:295676691
Protein name: Aldehyde Dehydrogenase [Burkholderia sp. CCGE1002]
SEQ ID NO: 26

| | |
|---|---|
| 1 | maissytdtr llingewcda asgktldvin patgqaigkv ahagipdldr aleaaqrgfe |
| 61 | awrkvpaner atimrkaaal vrerasdigr lmtgeggkpf aearvevlaa adiiewfade |
| 121 | grrvygrivp srnlaaqsqv lkepigpvaa ftpwnfpvnq vvrklsasla cgcsflvkap |
| 181 | eetpaspaal lqafveagvp pgtvglvfgd paeissylip hpvirkvtft gstpvgkqla |
| 241 | alagshmkra tmelgghapv ivaedadval avkaagaakf rnagqvcisp trflvhnsir |
| 301 | eefaaalvkh aeslklgdgl aegttlgpla narrisamar vvddarktga kvatggervg |
| 361 | tegnffaatv ltdvpleadv fnnepfgpva airgfdklee aiaeanrlpy glagyaytks |
| 421 | fanvhllsqr mevgmlwinq patptpempf ggvkdsgygs eggpeameay lvtkavtvms |
| 481 | v |

SeqID: YP_558960.1 GI:91783754
Protein name: 2,5-dioxopentanoate dehydrogenase (NAD+) [Burkholderia
xenovorans LB400]
SEQ ID NO: 27

| | |
|---|---|
| 1 | maipsytdtr llingewcda asgktldvin patgqaigkv ahagiadldr alaaaqrgfe |
| 61 | awrkvpaner attmrraaal vrerasdigr lmtqeqgkpf aearievlaa adiiewfade |
| 121 | grrvygrivp srnlaaqqlv lkepigpvaa ftpwnfpvnq vvrklsaala cgcsflvkap |
| 181 | eetpaspaal lqafveagvp agtvglvfgd paeisgylip hpvirkvtft gstpvgkqla |
| 241 | alagahmkra tmelgghapv ivaedadval avkaaggakf rnagqvcisp trflvhnsir |
| 301 | eefaaalvkh aeglklgdgl aegttlgpla narrlsamsk vlddarktga kvetggervg |
| 361 | segnffaptv ltnvsleadv fnnepfgpia airgfdklee aiaeanrlpy glagyaftks |
| 421 | fsnvhllsqg vevgmlwinq patpspempf ggvkdsgygs eggpeamegy lvtkaysvma |
| 481 | v |

SeqID: ZP_06846085.1 GI:296163325
Protein name: Aldehyde Dehydrogenase [Burkholderia sp. Ch1-1]
SEQ ID NO: 28

| | |
|---|---|
| 1 | maissytdtr llingewcda asgktldvvn patggaigkv ahagiadldr alaaaqrgfe |
| 61 | awrkvpaner attmrraaal vrerasdigr lmtigeqgkpf aearvevlaa adiiewfade |
| 121 | grrvygrivp srnlaaqqlv lkepigpvaa ftpwnfpvnq vvrklsaala cgcsflvkap |
| 181 | eetpaspaal lqafveagvp agtvglvfgd paeissylip hpvirkvtft gstpvgkqla |
| 241 | alagahmkra tmelgghapv ivaedadval avkaaggakf rnagqvcisp trflvhnsir |
| 301 | eefaaalvkh aeglklgdgl aegttlgpla narrltamsk vlddarktga kvetggervg |
| 361 | segnffaptv ltnvsleadv fnnepfgpia airgfdklee aiaeanrlpy glagyaftks |
| 421 | fsnvhllsqg levgmlwinq patpspempf ggvkdsgygs eggpeamegy lvtkaysvma |
| 481 | v |

SeqID:YP_001895827.1 GI:187924185
Protein name: aldehyde dehydrogenase [Burkholderia phytofirmans PsJN]
SEQ ID NO: 29

| | |
|---|---|
| 1 | matssytdtr llingewcda asgktldvin patgkaigkv ahagiadldr alaaaqrgfe |
| 61 | awrkvpaner attmrkaaal vrerasdigr lmtleggkpf aearievlaa adiiewfade |
| 121 | grrvygrivp srnlaaqqlv lkepigpvaa ftpwnfpvnq vvrklsaala cgcsflvkap |
| 181 | eetpaspaal lqafveagvp agtvglvfgd paeissylip hpvirkvtft gstpvgkqla |
| 241 | alagshmkra tmelgghapv ivaedadval avkaaggakf rnagqvcisp trflvhnsir |
| 301 | eefaaalvkh aeglklgdgl aegttlgpla narrltamsk vlddarktga kvetggervg |
| 361 | segnffaptv ltnvslesdv fnnepfgpia airgfdklee aiaeanrlpf glagyaftks |
| 421 | ftnvhllsqg levgmlwinq patpspempf ggvkdsgygs eggpeamegy lvtkaysvms |
| 481 | v |

SeqID: YP_003907074.1 GI:307729850
Protein name: aldehyde dehydrogenase [Burkholderia sp. CCGE1003]
SEQ ID NO: 30

| | |
|---|---|
| 1 | maissytdtr llingewcda asgktidvin patgqvigtv ahagiadldr aleaaqrgfe |
| 61 | awrkvpaher aavmrkaaal vrerasdigr lmtgeggkpf aeakievlaa adiiewfade |
| 121 | grrlygrvvp srnlaaqqlv lkepigpvaa ftpwnfpvnq ivrklsaala sgcsflvkap |
| 181 | eetpaspagl lqafveagvp agtvglvfgd paeisgylip hpvirkvtft gstpvgkqla |
| 241 | alagahmkra tmelgghapv ivaddadval avkaaggakf rnagqvcisp trflvhnsir |
| 301 | eefaaalvkh aeglklgdgl aegttlgpla narrltamsk vledarktga kvetggervg |
| 361 | segnffaptv ltnvsleadv fnnepfgpia airgfdklee aiaeanrlpf glagyaftks |
| 421 | fsnvhllsqq levgmlwinq patptpempf ggvkdsgygs eggpeameay lvtkavtvms |
| 481 | s |

SeqID: ZP_02887443.1 GI:170696312
Protein name: Aldehyde Dehydrogenase [Burkholderia graminis C4D1M]
SEQ ID NO: 31

| | |
|---|---|
| 1 | maissytdtr llingewcda asgktldvin patgqvigkv ahagiadldr aleaaqrgfe |
| 61 | awrkvpaher aavmrkaaal vrerasdigr lmtgeggkpf aeakvevlaa adiiewfade |
| 121 | grrlygrvvp srnlaaqqlv lkepigpvaa ftpwnfpvnq ivrklsaala sgcsflvkap |
| 181 | eetpaspagl lqafveagvp agtvglvfgd paeisnylip hpvirkvtft gstpvgkqla |
| 241 | slagahmkra tmelgghapv ivaedadval avkaaggakf rnagqvcisp trflvhnsir |
| 301 | eefaaalvkh aeglklgdgl adgttlgpla narrltamsk vlddarrtga kietggervg |
| 361 | tegnffaptv ltnvsleadv fnnepfgpia airgfdklee aiaeanrlpf glagyaftks |
| 421 | fanvhllsqg levgmlwinq patptpempf ggvkdsgygs eggpeameay lvtkavtvms |
| 481 | s |

SeqID: YP_001861563.1 GI:186474221
Protein name: aldehyde dehydrogenase [Burkholderia phymatum STM815]
SEQ ID NO: 32

| | |
|---|---|
| 1 | mvtsssytdt rllinnewcd aasgktldvv npatgkpigk vahagkadld raleaaqkgf |
| 61 | eawrkvpane rattmrkaag fvreradhia rlmtqeqgkp faearievls aadiiewfad |
| 121 | egrrvygrvv psrnlnaqsl vikepigpva aftpwnfpvn qvvrklsaal asgcsflvka |
| 181 | peetpaspaq llqafvdagv pagtvglvfg dpaeissyli phpvirkvtf tgstpvgkql |
| 241 | aalagshmkr atmelgghap vivaedadva lavkaaggak frnagqvcis ptrflvhnsi |
| 301 | reafaaalvk haeglkvgdg laegtqlgpl anarrltama siidnarstg atvatggeri |
| 361 | gsegnffapt vltdvplead vfnnepfgpi aairgfdnie daiaeanrlp fglagyaftk |
| 421 | sfrnvhllsq nlevgmlwin qpatptpemp fggvkdsgyg seggpeamea ylvtkavtvm |
| 481 | av |

SeqID: YP_004228054.1 GI:323525901
Protein name: aldehyde dehydrogenase [Burkholderia sp. CCGE1001]
SEQ ID NO: 33

| | |
|---|---|
| 1 | maissytdtr llingewcda asgktidvin patgqvigkv ahagiadldr aleaaqrgfe |
| 61 | awrkvpaher aavmrkaaal vrerasdigr lmtqeqgkpf aeakievlaa adiiewfade |
| 121 | grrlygrvvp srnlaaqqlv lkepigpvaa ftpwnfpvnq ivrklsaala sgcsflvkap |
| 181 | eetpaspagl lqafveagvp agtvglvfgd paeissylip hpvirkvtft gstpvgkqla |
| 241 | alagahmkra tmelgghapv ivaedadval avkaaggakf rnagqvcisp trflvhnsir |
| 301 | eefaaalvkh aeslklgdgl aegttlgpla narrltamsk vlddarktga kietggervg |
| 361 | segnffaptv ltnvsleadv fnnepfgpia airgfdklee aiaeanrlpf glagyaftks |
| 421 | fsnvhllsqq levgmlwinq patptpempf ggvkdsgygs eggpeameay lvtkavtvms |
| 481 | |

SeqID: YP_004361425.1 GI:330817720
Protein name: NAD-dependent aldehyde dehydrogenase [Burkholderia gladioli
BSR3]
SEQ ID NO: 34

| | |
|---|---|
| 1 | mtnttytdtq llingewcda esgktidvin patgkvigkv ahagiadldr aleaaqrgfe |
| 61 | twrkvtaydr aalmrkaaal vreradtiaq lmtqeqgkpl veakievlsa adiiewfade |
| 121 | grrvygrivp prnlavqqtv vkepvgpvaa ftpwnfpvnq vvrklsaala tgcsflvkap |
| 181 | eetpaspaql lrafvdagvp agvvglvygd paeisnylip hpvirkvtft gstpvgkqla |
| 241 | alagqhmkra tmelgghapv ivaedadldl avkaaggakf rnagqvcisp trflvhnsvr |
| 301 | edfakalvkh aeglkvgdgl algtnlgpla nsrrlgamek vvadarktga tvatggerig |
| 361 | segnffaptv ltdvpleadv fnnepfgpia airgfdsled aiteanrlpy glagyaftra |
| 421 | fknvhlltqr levgmlwinq patpwpempf ggvkdsgygs eggpealepy lvtksvtvta |
| 481 | a |

SeqID: YP_002912305.1 GI:238028074
Protein name: NAD-dependent aldehyde dehydrogenase [Burkholderia glumae
BGR1]
SEQ ID NO: 35

| | |
|---|---|
| 1 | mtntnytdtq llingewcda asgktldvvn patgqvigkv ahagiadldr aldaaqrgfe |
| 61 | twrkvsayer salmrkaaal vreransiaq lmtleqgkpl aearievlsa adiiewfade |
| 121 | grrvygrivp prnlavqqtv vkepvgpvaa ftpwnfpvnq vvrklsaala tgcsflvkap |
| 181 | eetpaspaql lrafvdagvp agvvglvygd paeisnylip hpvirkitft gstpvgkqla |
| 241 | alagqhmkra tmelgghapv ivaedadlel avkaaggakf rnagqvcisp trflvhnsvr |
| 301 | eafvkalvkh aeglkvgdgl eagtslgpla nprrltamek vvadarkaga tvatggerig |
| 361 | sagnffaptv ladvpldadv fnnepfgpva avrgfdsldd aiteanrlpy glagyaftrs |
| 421 | fknvhlltqr vevgmlwinq patpwpempf ggvkdsgygs eggpealepy lvtksvtvaa |
| 481 | v |

SeqID: ZP_04941711.1 GI:254248391
Protein name: Succinate-semialdehyde dehydrogenase (NAD(P)+) [Burkholderia
cenocepacia PC184]
SEQ ID NO: 36

| | |
|---|---|
| 1 | mnpatgkpig kvahagiadl dralaaagrg feawrkvpah eraatmrkaa alvreradai |
| 61 | aglmtgeggk pltearvevl saadiiewfa degrrvygri vpprnlnagq tvvkepvgpv |
| 121 | aaftpwnfpv nqvvrklsaa latgcsflvk apeetpaspa allrafvdag vpagviglvf |
| 181 | gdpaeissyl iphpvirkvt ftgstpvgkq laalagqhmk ratmelggha pvivaedadv |
| 241 | alavkaagga kfrnagqvci sptrflvhns irdeftralv khaeglkvgn gleegttlga |
| 301 | lanprrltam asvvdnarkv gasietgger igaegnffap tvianvplea dvfnnepfgp |
| 361 | vaairgfdkl edaiaeanrl pfglagyaft rsfanvhllt qrlevgmlwi nqpatpwpem |
| 421 | pfggvkdsgy gseggpeale pylvtksvtv may |

SeqID: ZP_02382650.1 GI:167590262
Protein name: Succinate-semialdehyde dehydrogenase (NAD(P)(+))
[Burkholderia ubonensis Bu]
SEQ ID NO: 37

| | |
|---|---|
| 1 | mahvtytdtq llingewtda asgktidvvn patgkaigkv ahagiadldr alaaacirgfe |
| 61 | qwrrvpaher aatmrkaaal vreradgiaq lmtgeggkpl vearlevlaa adiiewfade |
| 121 | grrvygrivp prnlgaqqtv vkepvgpvaa ftpwnfpvnq vvrklsaala tgcsflvkap |
| 181 | eetpaspaal lrafvdagvp agviglvygd paeisaylip hpvirkvtft gstpvgkhla |
| 241 | alagqhmkra tmelgghapv ivaedadval avkaaggakf rnagqvcisp trflvhnsir |
| 301 | deftralvkh aeglkvgngl degttlgala nprriaamts vvenaravga rvetggerig |
| 361 | tegnffaptv ladvpleadv fnnepfgpva airgfdsldd aiseanrlpy glagyaftrs |
| 421 | fanvhlltqr levgmlwinq patpwpempf ggvkdsgygs eggpealepy lvtksvtvma |
| 481 | v |

SeqID: YP_372358.1 GI:78062450
Protein name: 2,5-dioxopentanoate dehydrogenase (NAD+) [Burkholderia sp.
383]
SEQ ID NO: 38

| | |
|---|---|
| 1 | manvtytdtq llidgewvda asgktidvvn patgkpigkv ahagiadldr alaaaqrgfd |
| 61 | awrkvpaher aatmrkaaal vreradaiaq lmtgeggkpl tearvevlsa adiiewfade |
| 121 | grrvygrivp prnlgaqqtv vkepvgpvaa ftpwnfpvnq vvrklsaala tgcsflvkap |
| 181 | eetpaspaal lrafvdagvp agviglvygd paeissylip hpvirkvtft gstpvgkqla |
| 241 | amaglhmkra tmelgghapv ivaedadval avkaaggakf rnagqvcisp trflvhnsir |
| 301 | deftralvkh aeglkvgngl eegtalgala nprrltamas vvdnarkvga rietggerig |
| 361 | tegnffaptv iadvpleadv fnnepfgpva airgfdkldd aiaeanrlpf glagyaftrs |
| 421 | fanvhlltqr levgmlwinq patpwpempf ggvkdsgygs eggpealepy lvtksvtvma |
| 481 | v |

SeqED:ZP_04947381A GI:254254064
Protein name: NAD-dependent aldehyde dehydrogenase [Burkholderia dolosa
AUO158]
SEQ ID NO: 39

| | |
|---|---|
| 1 | mwmanvtytd tqllidgewv daasgktidv vnpatgkaig kvahagiadl dralaaaqrg |
| 61 | feawrkvpah eraatmrkaa alvreradti aglmtgeggk plaesrievl saadiiewfa |
| 121 | degrrvygri vpprnlgagq tvvkepvgpv aaftpwnfpv nqvvrklsaa latgcsflvk |
| 181 | apeetpaspa allrafvdag vpagviglvf gdpaeisayl iphpvirkvt ftgstpvgkq |
| 241 | laalagqhmk ratmelggha pvivaedadv alavkaagga kfrnagqvci sptrflvhns |
| 301 | irdeftralv khaeglkvgn gleegttlga lanprrltam asvvdnarkv garietgger |
| 361 | igsegnffap tviadvplea dvfnnepfgp vaairgfdkl ddaiaeanrl pyglagyaft |
| 421 | rsfanvhllt qrlevgmlwi nqpatpwpem pfggvkdsgy gseggpeale pylvtksvtv |
| 481 | may |

SeqID: BAE94276.1 GI:95102056
Protein name: alfa-ketoglutaric semialdehyde dehydrogenase [Azospirillum
brasilense]
SEQ ID NO: 40

| | |
|---|---|
| 1 | manvtytdtq llidgewvda asgktidvvn patgkpigry ahagiadldr alaaaqsgfe |
| 61 | awrkvpaher aatmrkaaal vreradaiaq lmtgeggkpl tearvevlsa adiiewfade |
| 121 | grrvygrivp prnlgaqqtv vkepvgpvaa ftpwnfpvnq vvrklsaala tgcsflvkap |
| 181 | eetpaspaal lrafvdagvp agviglvygd paeissylip hpvirkvtft gstpvgkqla |
| 241 | slaglhmkra tmelgghapv ivaedadval avkaaggakf rnagqvcisp trflvhnsir |
| 301 | deftralvkh aeglkvgngl eegttlgala nprrltamas vidnarkvga sietggerig |
| 361 | segnffaptv ianvpldadv fnnepfgpva airgfdklee aiaeanrlpf glagyaftrs |
| 421 | fanvhlltqr levgmlwinq patpwpempf ggvkdsgygs eggpealepy lvtksvtvma |
| 481 | v |

SeqID: ZP_03583019.1 GI:221210038
Protein name: succinate-semialdehyde dehydrogenase [NADP+] (ssdh)
[Burkholderia multivorans CGD1]
SEQ ID NO: 41

| | |
|---|---|
| 1 | manvtytdtq llidgewvda asgktidvvn patgrvigkv ahagiadldr alaaaqrgfe |
| 61 | awrkvpaher aatmrkaaal vreradtiaq lmtgeggkpl tearievlsa adiiewfade |
| 121 | grrvygrivp prnlgaqqtv vkepvgpvaa ftpwnfpvnq vvrklsaala tgcsflvkap |
| 181 | eetpaspaal lrafvdagvp agviglvygd paeissyvip hpvirkvtft gstpvgkqla |
| 241 | alaggnmkra tmelgghapv ivaedadval avkaaggakf rnagqvcisp trflvhnsir |
| 301 | deftralvkh aeglkvgngl eegttlgala nprrltamas vvenarkvga svetggerig |
| 361 | segnffaptv lanvpleadv fnnepfgpva airgfdkled aiaeanrlpy glagyaftrs |
| 421 | fanvhlltqr levgmlwinq patpwpempf ggvkdsgygs eggpealepy lvtksvtvma |
| 481 | v |

SeqID: YP_001779559.1 GI:170738299
Protein name: aldehyde dehydrogenase [Burkholderia cenocepacia MC0-3]
SEQ ID NO: 42

| | |
|---|---|
| 1 | manvtytdtq llidgewvda asgktidvvn patgkpigkv ahasiadldr alaaaqrgfe |
| 61 | awrkvpaher aatmrkaaal vreradtiaq lmtqeqgkpl tearvevlsa adiiewfade |
| 121 | grrvygrivp prnlgaqqtv vkepvgpvaa ftpwnfpvnq vvrklsaala tgcsflvkap |
| 181 | eetpaspaal lrafvdagvp agviglvfgd paeissylip hpvirkvtft gstpvgkqla |
| 241 | alagqhmkra tmelgghapv ivaedadval avkaaggakf rnagqvcisp trflvhnsir |
| 301 | deftralvkh aeglkvgngl eegttlgala nprrltamas vvdnarkvga sietggerig |
| 361 | aegnffaptv ianvpleadv fnnepfgpva airgfdkled aiaeanrlpf glagyaftrs |
| 421 | fanvhlltqr levgmlwinq patpwpempf ggvkdsgygs eggpealepy lvtksvtvma |
| 481 | v |

SeqID: YP_001584188.1 GI:161520761
Protein name: aldehyde dehydrogenase [Burkholderia multivorans ATCC 17616]
SEQ ID NO: 43

| | |
|---|---|
| 1 | manvtytdtq llidgewvda asgktidvvn patgkvigkv ahagiadldr alaaaqrgfe |
| 61 | awrkvpaher aatmrkaaar vreradtiaq lmtqeqgkpl tearievlsa adiiewfade |
| 121 | grrvygrivp prnlgaqqtv vkepvgpvaa ftpwnfpvnq vvrklsaala tgcsflvkap |
| 181 | eetpaspaal lrafvdagvp agviglvygd paeissyvip hpvirkvtft gstpvgkqla |
| 241 | alagqhmkra tmelgghapv ivaedadval avkaaggakf rnagqvcisp trflvhnsir |
| 301 | deftralvkh aeglkvgngl eegttlgala nprrltamas vvenarkvga svetggerig |
| 361 | segnffaptv lanvpleadv fnnepfgpva airgfdkled aiaeanrlpy glagyaftrs |
| 421 | fanvhlltqr levgmlwinq patpwpempf ggvkdsgygs eggpealepy lvtksvtvma |
| 481 | v |

SeqID: EGD03606.1 GI:325525897
Protein name: NADP-dependent succinate-semialdehyde dehydrogenase
[Burkholderia sp. TJI49]
SEQ ID NO: 44

| | |
|---|---|
| 1 | manvtytdtq llidgewvda asgktidvmn patgkvigkv ahagiadldr alaaaqrgfe |
| 61 | awrkvpaher aatmrkaaal vreradaiaq lmtqeqgkpl aearievlsa adiiewfade |
| 121 | grrvygrivp prnlgaqqtv vkepvgpvaa ftpwnfpvnq vvrklsaala tgcsflvkap |
| 181 | eetpaspaal lrafvdagvp agviglvygd paeissylip hpvirkvtft gstpvgkqla |
| 241 | alagqhmkra tmelgghapv ivaedadval avkaaggakf rnagqvcisp trflvhnsir |
| 301 | deftralvkh aeglkvgngl eegttlgala nprrltamas vvdnarkvga svetggerig |
| 361 | segnffaptv lanvpleadv fnnepfgpva airgfdkled aiaeanrlpy glagyaftrs |
| 421 | fanvhlltqr levgmlwinq patpwpempf ggvkdsgygs eggpealepy lvtksvtvma |
| 481 | v |

SeqID: YP_002234153.1 GI:206563390
Protein name: putative aldehyde dehydrogenase [Burkholderia cenocepacia
J2315]
SEQ ID NO: 45

| | |
|---|---|
| 1 | manvtytdtq llidgewvda asgktidvvn patgkpigkv ahagiadldr alaaaqrgfe |
| 61 | awrkvpaher aatmrkaaal vreradtiaq lmtqeqgkpl tearvevlsa adiiewfade |
| 121 | grrvygrivp prnlgaqqtv vkepvgpvaa ftpwnfpvnq vvrklsaala tgcsflvkap |
| 181 | eetpaspaal lrafvdagvp agviglvygd paeissylip hpvirkvtft gstpvgkqla |
| 241 | alagqhmkra tmelgghapv ivaedadval avkaaggakf rnagqvcisp trflvhnsir |
| 301 | deftralvkh aeglkvgngl eegttlgala nprrltamas vvdnarkvga sietggerig |
| 361 | aegnffaptv ianvpleadv fnnepfgpva airgfdklee aiaeanrlpf glagyaftrs |
| 421 | fanvhlltqr levgmlwinq patpwpempf ggvkdsgygs eggpealepy lvtksvtvma |
| 481 | v |

SeqID: ZP_03569460.1 GI:221196413
Protein name: succinate-semialdehyde dehydrogenase [NADP+] (ssdh) [Burkholderia
multivorans CGD2M]
SEQ ID NO: 46

| | |
|---|---|
| 1 | manvtytdtq llidgewvda asgktidvvn patgkvigkv ahagiadldr alaaaqrgfe |
| 61 | awrkvpaher aatmrkaaal vreradtiaq lmtqeqgkpl tearievlsa adiiewfade |
| 121 | grrvygrivp prnlgaqqtv vkepvgpvaa ftpwnfpvnq vvrklsaala tgcsflvkap |
| 181 | eetpaspaal lrafvdagvp agviglvygd paeissylip hpvirkvtft gstpvgkqla |
| 241 | alagqhmkra tmelgghapv ivaedadval avkaaggakf rnagqvcisp trflvhnsir |
| 301 | deftralvkh aeglkvgngl eegttlgala nprrltamas vvenarkvga svetggerig |
| 361 | segnffaptv lanvpleadv fnnepfgpva airgfdkled aiaeanrlpy glagyaftrs |
| 421 | fanvhlltqr levgmlwinq patpwpempf ggvkdsgygs eggpealepy lvtksvtvma |
| 481 | v |

SeqID: YP_001117385.1 GI:134293649
Protein name: 2,5-dioxopentanoate dehydrogenase (NAD+) [Burkholderia
vietnamiensis G4]
SEQ ID NO: 47

| | |
|---|---|
| 1 | manvtytdtq llidgewvda asgktidvvn patgkaigkv ahagiadldr alaaaqrgfe |
| 61 | awrkvpaher aatmrkaaal vreradaiaq lmtqeqgkpl tearievlsa adiiewfade |
| 121 | grrvygrivp prnlgaqqmv vkepvgpvaa ftpwnfpvnq vvrklcaala tgcsflvkap |
| 181 | eetpaspaal lrafvdagvp agvvglvygd paeissylip hpvirkvtft gstpvgkqla |
| 241 | alagqhmkra tmelgghapv ivaedadval avkaaggakf rnagqvcisp trflvhnsir |
| 301 | deftralvah aqglkigngl degttlgala nprrltamas vvenarkvga sietggerig |
| 361 | segnffaptv ianvpleadv fnnepfgpva airgfdkled aiseanrlpf glagyaftrs |
| 421 | fanvhlltqr levgmlwinq patpwpempf ggvkdsgygs eggpealepy lvtksvtvma |
| 481 | v |

SeqID: YP_623820.1 GI:107026309
Protein name: succinate-semialdehyde dehydrogenase (NAD(P)+) [Burkholderia
cenocepacia AU 1054]
SEQ ID NO: 48

| | |
|---|---|
| 1 | manvtytdtq llidgewvda asgktidvvn patgkpigkv ahagiadldr alaavqrgfe |
| 61 | awrkvpaher aatmrkaaal vreradtiaq lmtqeqgkpl tearvevlsa adiiewfade |
| 121 | grrvygrivp prnfnaqqtv vkepvgpvaa ftpwnfpvnq vvrklsaala tgcsflvkap |
| 181 | eetpaspaal lrafvdagvp agviglvfgd paeissylip hpvirkvtft gstpvgkqla |
| 241 | alagqhmkra tmelgghapv ivaedadval avkaaggakf rnagqvcisp trflvhnsir |
| 301 | deftralvkh aeglkvgngl eegttlgala nprrltamas vvdnarkvga sietggerig |
| 361 | aegnffaptv ianvpleadv fnnepfgpva airgfdkled aiaeanrlpf glagyaftrs |
| 421 | fanvhlltqr levgmlwinq patpwpempf ggvkdsgygs eggpealepy lvtksvtvma |
| 481 | v |

SeqID: ZP_02891604.1 GI:170700605
Protein name: Aldehyde Dehydrogenase [Burkholderia ambifaria IOP40-10]
SEQ ID NO: 49

| | |
|---|---|
| 1 | manvtytdtq llidgewvda asgktidvvn patgkaigkv ahagiadldr alaaaqrgfe |
| 61 | awrkvpaner aatmrkaaal vreradtiaq lmtgeggkpl tearvevlsa adiiewfade |
| 121 | grrvygrivp prnlgaqqmv vkepvgpvaa ftpwnfpvnq vvrklsaala tgcsflvkap |
| 181 | eetpaspaal lrafvdagvp agviglvygd paeissylia hpvirkvtft gstpvgkqla |
| 241 | alagqhmkra tmelgghapv ivaedadval avkaaggakf rnagqvcisp trflvhnsir |
| 301 | deftralvqh aeglkigngl eegttlgala nprrltamvs vvdnarkvga rietggerig |
| 361 | segnffaptv ianvpleadv fnnepfgpva airgfdkldd aiaeanrlpf glagyaftrs |
| 421 | fanvhlltgr levgmlwinq patpwpempf ggvkdsgygs eggpealepy lvtksvtvma |
| 481 | v |

SeqID: YP_001810977.1 GI:172063326
Protein name: aldehyde dehydrogenase [Burkholderia ambifaria MC40-6]
SEQ ID NO: 50

| | |
|---|---|
| 1 | manvtytdtq llidgewvda asgktidvvn patgkaigkv ahagiadldr alvaaqrgfe |
| 61 | awrkvpaner aatmrkaaal vreradtiaq lmtgeggkpl tearvevlsa adiiewfade |
| 121 | grrvygrivp prnlgaqqmv vkepvgpvaa ftpwnfpvnq vvrklsaala tgcsflvkap |
| 181 | eetpaspaal lrafvdagvp agviglvygd paeissylia hpvirkvtft gstpvgkqla |
| 241 | alagqhmkra tmelgghapv ivaedadval avkaaggakf rnagqvcisp trflvhnsir |
| 301 | deftralvqh aeglkigngl eegttlgala nprrltamas vvdnarkvga sietggerig |
| 361 | segnffaptv ianvpleadv fnnepfgpva airgfdkled aiaeanrlpf glagyaftrs |
| 421 | fanvhlltgr levgmlwinq patpwpempf ggvkdsgygs eggpealepy lvtksvtvma |
| 481 | v |

SeqID: ZP_02911594.1 GI:171322894
Protein name: Aldehyde Dehydrogenase_[Burkholderia ambifaria MEX-5]
SEQ ID NO: 51

| | |
|---|---|
| 1 | manvtytdtq llidgewvda asgrtidvvn patgkaigkv ahagiadldr alaaaqrgfe |
| 61 | awrkvpaner aatmrkaaal vreradaiaq lmtgeggkpl tearvevlsa adiiewfade |
| 121 | grrvygrivp prnlgaqqmv vkepvgpvaa ftpwnfpvnq vvrklsaala tgcsflvkap |
| 181 | eetpaspaal lrafvdagvp agviglvygd paeissylia hpvirkvtft gstpvgkqla |
| 241 | alagqhmkra tmelgghapv ivaedadval avkaaggakf rnagqvcisp trflvhnsir |
| 301 | deftralvqh aeglkigngl eegttlgala nprrltamas vvenarkvga sietggerig |
| 361 | segnffaptv ianvpleadv fnnepfgpva airgfdkled aiaeanrlpf glagyaftrs |
| 421 | fanvhlltqr levgmlwinq patpwpempf ggvkdsgygs eggpealepy lvtksvtvma |
| 481 | v |

SeqID: YP_775718.1 GI:115358580
Protein name: succinate-semialdehyde dehydrogenase (NAD(P)(+)) [Burkholderia
ambifaria AMMD]
SEQ ID NO: 52

| | |
|---|---|
| 1 | manvtytdtq llidgewvda asgktidvvn patgkaigkv ahagiadldr alaaaqrgfe |
| 61 | awrkvpaner aatmrkaaal vreradaiaq lmtgeggkpl tearvevlsa adiiewfade |
| 121 | grrvygrivp prnlgaqqmv vkepvgpvaa ftpwnfpvnq vvrklsaala tgcsflvkap |
| 181 | eetpaspaal lrafvdagvp agviglvyge paeissylia hpvirkvtft gstpvgkqla |
| 241 | alagqhmkra tmelgghapv ivaedadval avkaaggakf rnagqvcisp trflvhnsir |
| 301 | deftralvqh aeglkigngl eegttlgala nprrltamas vvenarkvga sietggerig |
| 361 | segnffaptv ianvpleadv fnnepfgpva airgfdkled aiaeanrlpf glagyaftrs |
| 421 | fanvhllsqr levgmlwinq patpwpempf ggvkdsgygs eggpealepy lvtksvtvma |
| 481 | v |

SeqID: NP_015264.1 GI:6325196
Protein name: Aldehyde dehydrogenase, ALD6 [Saccharomyces cerevisiae]
SEQ ID NO: 53

| | |
|---|---|
| 1 | mtklhfdtae pvkitlpngl tyeqptglfi nnkfmkaqdg ktypvedpst entvcevssa |
| 61 | ttedveyaie cadrafhdte watqdprerg rllskladel esqidlvssi ealdngktla |
| 121 | largdvtiai nclrdaaaya dkvngrtint gdgymnfttl epigvcgqii pwnfpimmla |
| 181 | wkiapalamg nvcilkpaav tpinalyfas lckkvgipag vvnivpgpgr tvgaaltndp |
| 241 | rirklaftgs tevgksvavd ssesnlkkit lelggksahl vfddanikkt lpnlvngifk |
| 301 | nagqicssgs riyvgegiyd ellaafkayl eteikvgnpf dkanfqgait nrqqfdtimn |
| 361 | yidigkkega kiltggekvg dkgyfirptv fydvnedmri vkeeifgpvv tvakfktlee |
| 421 | gvemanssef glgsgietes lstglkvakm lkagtvwint yndfdsrvpf ggvkqsgygr |
| 481 | emgeevyhay tevkavrikl |

SeqID: NP_013893.1 GI:6323822
Protein name: Aldehyde dehydrogenase, ALD2 [Saccharomyces cerevisiae]
SEQ ID NO: 54

| | |
|---|---|
| 1 | mptlytdiei pqlkislkqp lglfinnefc pssdgktiet vnpatgepit sfqaanekdv |
| 61 | dkavkaaraa fdnvwsktss eqrgiylsnl lklieeeqdt laaletldag kpyhsnakgd |
| 121 | lagilqltry fagsadkfdk gatipltfnk faytlkvpfg vvaqivpwny plamacwklq |
| 181 | galaagntvi ikpaentsls llyfatlikk agfppgvvni vpgygslvgq alashmdidk |
| 241 | isftgstkvg gfvleasgqs nlkdvtlecg gkspalvfed adldkaidwi aagifynsgq |
| 301 | nctansrvyv gssiydkfve kfketakkew dvagkfdpfd ekcivgpvis stqydriksy |
| 361 | iergkreekl dmfqtsefpi ggakgyfipp tiftdvpqts kllqdeifgp vvvvskftny |
| 421 | ddalklandt cyglasavft kdvkkahmfa rdikagtvwi nssndedvtv pfggfkmsgi |
| 481 | grelgqsgvd tylqtkavhi nlsldn |

SeqID: NP_013892.1 GI:6323821
Protein name: Aldehyde dehydrogenase, ALD3 [Saccharomyces cerevisiae]
SEQ ID NO: 55

| | |
|---|---|
| 1 | mptlytdiei pqlkislkqp lglfinnefc pssdgktiet vnpatgepit sfqaanekdv |
| 61 | dkavkaaraa fdnvwsktss eqrgiylsnl lklieeeqdt laaletldag kpfhsnakqd |
| 121 | laqiieltry yagavdkfnm getipltfnk faytlkvpfg vvagivpwny plamacrkmq |
| 181 | galaagntvi ikpaentsls llyfatlikk agfppgvvnv ipgygsvvgk algthmdidk |
| 241 | isftgstkvg gsvleasgqs nlkditlecg gkspalvfed adldkaiewv angiffnsgq |
| 301 | ictansrvyv qssiydkfve kfketakkew dvagkfdpfd ekcivgpvis stqydriksy |
| 361 | iergkkeekl dmfqtsefpi ggakgyfipp tiftdvpets kllrdeifgp vvvvskftny |
| 421 | ddalklandt cyglasavft kdvkkahmfa rdikagtvwi nqtncieeak vpfggfkmsgi |
| 481 | gresgdtgvd nylqiksvhv dlsldk |

1. A recombinant cell modified to exhibit increased biosynthesis of pentanoic acid compared to a wild-type control.

2. A recombinant microbial cell modified to exhibit increased biosynthesis of 2-methylbutyric acid compared to a wild-type control.

3. The recombinant microbial cell of claim 1 wherein the microbial cell is a fungal cell.

4-5. (canceled)

6. The recombinant cell of claim 1 wherein the microbial cell is a bacterial cell.

7-19. (canceled)

20. The recombinant cell of claim 1 wherein the microbial cell is photosynthetic.

21. The recombinant cell of claim 1 wherein the microbial cell is cellulolytic.

22. The recombinant cell of claim 1 wherein the increased biosynthesis of pentanoic acid compared to a wild-type control comprises an increase in conversion of L-aspartate to L-threonine compared to a wild-type control, an increase in conversion of L-threonine to 2-ketobutyrate compared to a wild-type control, an increase in 2-ketobutyrate elongation activity compared to a wild-type control, an increase in 2-ketovalerate elongation activity compared to a wild-type control, an increase in ketoacid decarboxylase activity compared to a wild-type control, an increase in ketoacid decarboxylase selectivity toward a predetermined substrate compared to a wild-type control, or an increase in aldehyde dehydrogenase activity compared to a wild-type control.

23. The recombinant cell of claim 2 wherein the increased biosynthesis of 2-methylbutyric acid compared to a wild-type control comprises an increase in conversion of L-aspartate to L-threonine compared to a wild-type control, an increase in conversion of L-threonine to 2-ketobutyrate compared to a wild-type control, an increase in conversion of 2-ketobutyrate to 2-keto-3-methylvalerate, an increase in ketoacid decarboxylase activity compared to a wild-type control, an increase in ketoacid decarboxylase selectivity toward a predetermined substrate compared to a wild-type control, or an increase in aldehyde dehydrogenase activity compared to a wild-type control.

24. A method comprising:
Incubating the recombinant cell of claim 1 in medium that comprises a carbon source under conditions effective for the recombinant cell to produce pentanoic acid, wherein the carbon source comprises one or more of: glucose, pyruvate, L-aspartate, L-threonine, 2-ketobutyrate, 2-ketovalerate, 2-ketocaproate, valeraldehyde, CO₂, cellulose, xylose, sucrose, arabinose, or glycerol.

25. A method comprising:
incubating the recombinant cell of claim 2 in medium that comprises a carbon source under conditions effective for the recombinant cell to produce 2-methylbutyric acid, wherein the carbon source comprises one or more of: glucose, pyruvate, L-aspartate, L-threonine, 2-ketobutyrate, 2-keto-3-methylvalerate, 2-methyl butyraldehyde, CO₂, cellulose, xylose, sucrose, arabinose, or glycerol.

26. A method comprising:
introducing into a host cell a heterologous polynucleotide encoding at least one polypeptide that catalyzes conversion of a carbon source to pentanoic acid, wherein the at least one polynucleotide is operably linked to a promoter so that the modified host cell catalyzes conversion of the carbon source to pentanoic acid.

27. The method of claim 26 wherein the carbon source comprises one or more of: glucose, pyruvate, L-aspartate, L-threonine, 2-ketobutyrate, 2-ketovalerate, 2-ketocaproate, valeraldehyde, CO₂, cellulose, xylose, sucrose, arabinose, or glycerol.

28. A method comprising:
introducing into a host cell a heterologous polynucleotide encoding at least one polypeptide that catalyzes conversion of a carbon source to 2-methylbutyric acid, wherein the at least one polynucleotide is operably linked to a promoter so that the modified host cell catalyzes conversion of the carbon source to 2-methylbutyric acid.

29. The method of claim 28 wherein the carbon source comprises one or more of: glucose, pyruvate, L-aspartate, L-threonine, 2-ketobutyrate, 2-keto-3-methylvalerate, 2-methyl butyraldehyde, CO₂, cellulose, xylose, sucrose, arabinose, or glycerol.

30. The method of claim 24 wherein the host cell is a fungal cell.

31-32. (canceled)

33. The method of claim 24 wherein the host cell is a bacterial cell.

34-46. (canceled)

47. The method of claim 24 wherein the host cell is photosynthetic.

48. The method of claim 24 wherein the host cell is cellulolytic.

49. The recombinant microbial cell of claim 2 wherein the microbial cell is a fungal cell.

50. The recombinant cell of claim 2 wherein the microbial cell is a bacterial cell.

51. The recombinant cell of claim 2 wherein the microbial cell is photosynthetic.

52. The recombinant cell of claim 2 wherein the microbial cell is cellulolytic.

53. The method of claim 25 wherein the host cell is a fungal cell.

54. The method of claim 25 wherein the host cell is a bacterial cell.

55. The method of claim 25 wherein the host cell is photosynthetic.

56. The method of claim 25 wherein the host cell is cellulolytic.

57. The method of claim 26 wherein the host cell is a fungal cell.

58. The method of claim 26 wherein the host cell is a bacterial cell.

59. The method of claim 26 wherein the host cell is photosynthetic.

60. The method of claim 26 wherein the host cell is cellulolytic.

61. The method of claim 28 wherein the host cell is a fungal cell.

62. The method of claim 28 wherein the host cell is a bacterial cell.

63. The method of claim 28 wherein the host cell is photosynthetic.

64. The method of claim 28 wherein the host cell is cellulolytic.


SEQUENCE LISTING (55 sequences)

SEQ ID NO: 1 - protein, 550 aa, Salmonella bongori
MQTPYTVADYLLDRLAGCGIDHLFGVPGDYNLQFLDHVIDHPTLRWVGCANELNAAYAADGYARMSGAGALLTTFGVGELSAINGIAGSYAEYVPVLHIVGAPCSDAQQRGELMHHTLGDGDFRHFYRMSQAISAASAVLNEQNACYEIDRVLGEMLTAHRPCYILLPADVAKKPAIPPTETLMLPANKAQSSVETAFRYHARQCLMNSRRIALLADFLARRFGLRPLLQRWMVETPIAHATLLMGKGLFNEQHPNFVGTYSAGASSKEVRQAIEDADMVICVGTRFVDTLTAGFTQQLPAERTLEIQPYASRIGDSWFTLPMELAVSILRELCLECAFAPSPTRSSGQSIPVEKGALTQENFWQTLQQFIKPGDIILVDQGTAAFGAAALSLPDGAEVLVQPLWGSIGYSLPAAFGAQTACPDRRVILIIGDGAAQLTIQEMGSMLRDEQAPIILLLNNEGYTVERAIHGAAQRYNDIASWNWTQIPQALSAAQQAECWRVTQAIQLEEILARLARPQRLSLIEVMLPKADLPELLRTVTRALEMRNGG

SEQ ID NO: 2 - protein, 516 aa, Salmonella enterica
LDHVIDHPTLRWVGCANELNAAYTADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTEALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQCLKPGDIILVDQGTAAFGAAALSLPDGAEVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 3 - protein, 550 aa, Salmonella enterica
MQTPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFHHFYRMSQAISAGSAILNEQNACFEIDRVLGEMVAARRPGYIMLPADVAKKTAIPPIEALTLPAHETQNGVETAFRYRARQCLMNSRRIALLADFLARRFGLRPLLQRWMAETSIAHATLLMGKGLFDEQHPNFVGTYSAGASSKAVRQAIEDADMVICVGTRFVDTLTAGFTQQLPAERTLEIQPYASRIGETWFNLPMAQAVSTLRELCLECAFAPPPTRPVCQPVQIEKGELTQENFWQTLQQYLKPGDIILVDQGTAAFGAAALSLPDGAEVVVQPLWGSIGYSLPAAFGAQTACPDRRVILIIGDGAAQLTIQEMGSMLRDGQAPIILLLNNDGYTVERAIHGAAQRYNDIASWNWTQIPQALNAAQQAECWRVTQAIQLAEVLERLARPQRLSFIEVMLPKADLPELLRTVTRALEARNGG

SEQ ID NO: 4 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALTLPVHEAQSGVETAFRYHARQCLMNSRRIALLADFLAGRFGLRPLLQRWMAETPIAHATLLMEKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIVLVDQGTAAFGAAALSLPDGAEVVVQPLWGSIGYSLPAAFGAQTACPDRRVILIIGDGAAQLTIQEMGSMLRDGQAPVILLLNNGGYTVERAIHGAAQRYNDIASWNWTQIPPALNAAQQVECWRVAQAIQLAEVLERLARPQRLSFIEVMLPKADLPELLRTVTRALEARNGG

SEQ ID NO: 5 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISVASAILDEQNACFEIDRVLGEMFAARRPGYIMLPADVAKKTAIPPTQALTLPVHEAQSGVETAFRYHARQCLMNSRRIALLADFLAGRFGLRPLLQRWMVETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIVLVDQGTAAFGAAALSLPDGAEVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLVRPQRLSFIEVMLPKADLPELLRTVTRALEARNGG

SEQ ID NO: 6 - protein, 549 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISVASAILYEQNACFEIDRVLGEMLAARRPGYIMLPADVAKKTAIPPTEALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSTGQPVRIDKGELTQESFWQTLQQYLKPGDIILVDQGTAAFGAAALSLPDGAEVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNG

SEQ ID NO: 7 - protein, 550 aa, Salmonella enterica
MQNPYTVADYLLDRLAGCGIGHLFGVPGDYNLQFLDHVIDHPTLRWVGCANELNAAYTADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTEALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQCLKPGDIILVDQGTAAFGAAALSLPDGAEVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 8 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISVASAILDEQNACFEIDRVLGEMLVARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTVGFTQQLPTERTLEIQPYASRIGETWFNLPMAQAVSTLRELCLECAFAPPPTRSAGQPVRIDKGELTQESFWQTLQQYLKPGDIILVDQGTAAFGAAALSLPDGAKVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 9 - protein, 550 aa, Salmonella enterica
MQNPYTVADYLLDRLAGCGIGHLFGVPGDYNLQFLDHVIDHPTLRWVGCANELNAAYAADGYARMSGTGALLTTFGVGELSAINGIAGSYAEYVPVLHIVGAPCSAAQQRGELMHHTLGDGDFRHFYRMSQAISVASSILDEQNACFEIDRVLGEMLAARRPGYIMLPADVAKKTAIPPTEALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 10 - protein, 550 aa, Salmonella enterica
MQNPYTVADYLLDRLAGCGIGHLFGVPGDYNLQFLDHVIDHPTLRWVGCANELNAAYAADGYARMSGTGALLTTFGVGELSAINGIAGSYAEYVPVLHIVGAPCSAAQQRGELMHHTLGDGDFRHFYRMSQAISVASSILDEQNACFEIDRVLGEMLAARRPGYIMLPADVAKKTAIPPTEALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVVQPLWGSIGYSLPAAFGAQTACPDRRVILVIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 11 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPTERTLEIQPYALRIGETWFNLPMAQAVSTLRELCLECAFAPPPTRSAGQPVRIDKGELTQESFWQTLQQYLKPGDIILVDQGTAAFGAAALSLPDGAEVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKAELPELLRTVTRALEARNGG

SEQ ID NO: 12 - protein, 550 aa, Salmonella enterica
MQNPYTVADYLLDRLAGCGIGHLFGVPGDYNLQFLDHVIDHPTLRWVGCANELNAAYTADGYARMSGAGALLTTFGVGELSAINGIAGSYAEYVPVLHIVGAPCSAAQQRGELMHHTLGDGDFRHFYRMSQAISVASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQGIQLAEVLERLARPQRLSFIEVMLPKADLPELLRTVTRALEARNGG

SEQ ID NO: 13 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPTERTLEIQPYASRIGETWFNLPMAQAVSTLRELCLECAFAPPPTRSAGQPVRIDKGELTQESFWQTLQQYLKPGDIILVDQGTAAFGAAALSLPDGAKVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 14 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPTERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVVQPLWGSIGYSLPAAFGAQTACPDRRVILIIGDGAAQLTIQEMGSMLRDGQAPVILLLNNDGYTVERAIHGAARRYNDIASWNWTQIPPALNAAQQAECWRVTQAIQLAEVLERLARPQRLSFIEVMLPKADLPELLRTVTRALEARNGG

SEQ ID NO: 15 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPTERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVVQPLWGAIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 16 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMFAARRPGYIMLPADVAKKTAIPPTQALTLPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIVLVDQGTAAFGAAALSLPDGAEVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 17 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTARFTQQLPAERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVLQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 18 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIVLVDQGTAAFGAAALSLPDGAEVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 19 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPTERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 20 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 21 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVLQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 22 - protein, 480 aa, Variovorax paradoxus
MTATYTDTRLLIDNEWVDATGGKTLDVVNPATGKVIGKVAHASIADLDRALAAAQRGFDKWRNTPANERAAVMRRAAGLIRERAGDIAKLLTQEQGKPLAEAKGETLAAADIIEWFADEGRRVYGRIVPSRNLAAQQLVLKEPLGPVAAFTPWNFPINQIVRKLGAALATGCSFLVKAPEETPASPAALLQAFVDAGIPPGTVGLVFGNPAEISNYLIAHPIIRKVTFTGSTPVGKQLAALAGSHMKRVTMELGGHAPVIVAEDADVALAVKAAGAAKFRNAGQVCISPTRFLVHNSLREEFARTLVKYTEGLKLGDGLAEGTTIGPLANARRLTAMAYVLEDARKKGATVAAGGERVGDSGNFFAPTVLTDVPLDADVFNNEPFGPIAAIRGFDTLEEAIAEANRLPFGLAGYAFTKSIKSAHLLSQKLELGMLWINQPATPSPEMPFGGVKDSGYGSEGGPEALEAYLNTKAVSILGV

SEQ ID NO: 23 - protein, 480 aa, Variovorax paradoxus
MTATYTDTRLLIDNEWVDATGGKTLDVVNPATGKAIGKVAHASIADLDRALAAAQRGFEKWRNTPANERAAVMRRAAGLIRERAPEIAKLLTQEQGKPLAEAKGETLAAADIIEWFADEGRRVYGRIVPSRNLAAQQLVIKEPLGPVAAFTPWNFPINQIVRKLGAALATGCSFLVKAPEETPASPAALLQAFVDAGIPPGTVGLVFGNPAEISNYLISHPIIRKVTFTGSTPVGKQLAALAGSHMKRVTMELGGHAPVIVAEDADVALAVKAAGAAKFRNAGQVCISPTRFLVHNSLREEFARTLVKYTEGLKLGDGLAEGTTLGPLANARRLTAMAHVLDDARKKGATVAAGGERVGDTGNFFAPTVLTDVPLDADVFNNEPFGPIAAIRGFDTLEEAIAEANRLPFGLAGYAFTRSIKNAHLLSQKLELGMLWINQPAAPSPEMPFGGVKDSGYGSEGGPEALEAYLNTKAVSIMSV

SEQ ID NO: 24 - protein, 481 aa, Burkholderia sp. H160
MAISSYTDTRLLINGEWCDAVSGKTLDVINPATGQAIGKVAHAGIADLDRALDAAQRGFEAWRKVPAHERATIMRKAAALVRERAADIGRLMTQEQGKPFAEARVEVLAAADIIEWFADEGRRVYGRIVPSRNLAAHSQVLKEPIGPVAAFTPWNFPVNQVVRKLSASLACGCSFLVKAPEETPASPAALLQAFVEAGVPPGTVGLVFGDPAEISSYLIPHPVIRKVTFTGSTPVGKQLAALAGTHMKRATMELGGHAPVIVAEDADVALAVKAAGAAKFRNAGQVCISPTRFLVHNSIREEFAAALVKHAESLKLGDGLAEGTTLGPLANARRLSAMAKVVEDARKTGAKVATGGERVGSEGNFFAATVLTDVPLEADVFNNEPFGPVAAIRGFDTLDEAITEANRLPYGLAGYAYTKSFANVHQLSQRMEVGMLWINQPATPTPEMPFGGVKDSGYGSEGGPEAMEAYLVTKAVTIMAV

SEQ ID NO: 25 - protein, 481 aa, Burkholderia rhizoxinica
MVTSSYTDTRLLIDGQWCDAASGKTLDVVNPATGQVIGRVAHAGIADLDRALAAAQRGFDTWRKVPVHERAATMRKAATLVRERAEGIARLMTQEQGKPFAEARIEVLSAADIIEWFADEGRRVYGRIVPSRNLAVQQSVLKEPIGPVAAFTPWNFPVNQVVRKLSAALACGCSFLVKAPEETPASPAGLLQAFVDAGVPAGTIGLVFGDPAAISSYLIAHPVIRKVTFTGSTPVGKQLAALAGAHMKRATMELGGHAPVIVAEDADIALAIKAAGGAKFRNAGQVCISPTRFLVHNSIREAFEAALVKHAQGLKLGDGLAQGTTLGPLANARRLTAMTRIVENARATGATVATGGERVGSAGNFFAPTVLTNVPRDADVFNQEPFGPVAAVRGFDRLEDAIAEANRLPYGLAGYAFTRSVRNVHLLSHQLEVGMLWINQPATPWPEMPFGGVKDSGYGSEGGPEAMEAYLVTKAVSVAAV

SEQ ID NO: 26 - protein, 481 aa, Burkholderia sp. CCGE1002
MAISSYTDTRLLINGEWCDAASGKTLDVINPATGQAIGKVAHAGIPDLDRALEAAQRGFEAWRKVPANERATIMRKAAALVRERASDIGRLMTQEQGKPFAEARVEVLAAADIIEWFADEGRRVYGRIVPSRNLAAQSQVLKEPIGPVAAFTPWNFPVNQVVRKLSASLACGCSFLVKAPEETPASPAALLQAFVEAGVPPGTVGLVFGDPAEISSYLIPHPVIRKVTFTGSTPVGKQLAALAGSHMKRATMELGGHAPVIVAEDADVALAVKAAGAAKFRNAGQVCISPTRFLVHNSIREEFAAALVKHAESLKLGDGLAEGTTLGPLANARRLSAMARVVDDARKTGAKVATGGERVGTEGNFFAATVLTDVPLEADVFNNEPFGPVAAIRGFDKLEEAIAEANRLPYGLAGYAYTKSFANVHLLSQRMEVGMLWINQPATPTPEMPFGGVKDSGYGSEGGPEAMEAYLVTKAVTVMSV

SEQ ID NO: 27 - protein, 481 aa, Burkholderia xenovorans
MAIPSYTDTRLLINGEWCDAASGKTLDVINPATGQAIGKVAHAGIADLDRALAAAQRGFEAWRKVPANERATTMRRAAALVRERASDIGRLMTQEQGKPFAEARIEVLAAADIIEWFADEGRRVYGRIVPSRNLAAQQLVLKEPIGPVAAFTPWNFPVNQVVRKLSAALACGCSFLVKAPEETPASPAALLQAFVEAGVPAGTVGLVFGDPAEISGYLIPHPVIRKVTFTGSTPVGKQLAALAGAHMKRATMELGGHAPVIVAEDADVALAVKAAGGAKFRNAGQVCISPTRFLVHNSIREEFAAALVKHAEGLKLGDGLAEGTTLGPLANARRLSAMSKVLDDARKTGAKVETGGERVGSEGNFFAPTVLTNVSLEADVFNNEPFGPIAAIRGFDKLEEAIAEANRLPYGLAGYAFTKSFSNVHLLSQQVEVGMLWINQPATPSPEMPFGGVKDSGYGSEGGPEAMEGYLVTKAVSVMAV

SEQ ID NO: 28 - protein, 481 aa, Burkholderia sp. Ch1-1
MAISSYTDTRLLINGEWCDAASGKTLDVVNPATGQAIGKVAHAGIADLDRALAAAQRGFEAWRKVPANERATTMRRAAALVRERASDIGRLMTQEQGKPFAEARVEVLAAADIIEWFADEGRRVYGRIVPSRNLAAQQLVLKEPIGPVAAFTPWNFPVNQVVRKLSAALACGCSFLVKAPEETPASPAALLQAFVEAGVPAGTVGLVFGDPAEISSYLIPHPVIRKVTFTGSTPVGKQLAALAGAHMKRATMELGGHAPVIVAEDADVALAVKAAGGAKFRNAGQVCISPTRFLVHNSIREEFAAALVKHAEGLKLGDGLAEGTTLGPLANARRLTAMSKVLDDARKTGAKVETGGERVGSEGNFFAPTVLTNVSLEADVFNNEPFGPIAAIRGFDKLEEAIAEANRLPYGLAGYAFTKSFSNVHLLSQQLEVGMLWINQPATPSPEMPFGGVKDSGYGSEGGPEAMEGYLVTKAVSVMAV

SEQ ID NO: 29 - protein, 481 aa, Burkholderia phytofirmans
MATSSYTDTRLLINGEWCDAASGKTLDVINPATGKAIGKVAHAGIADLDRALAAAQRGFEAWRKVPANERATTMRKAAALVRERASDIGRLMTLEQGKPFAEARIEVLAAADIIEWFADEGRRVYGRIVPSRNLAAQQLVLKEPIGPVAAFTPWNFPVNQVVRKLSAALACGCSFLVKAPEETPASPAALLQAFVEAGVPAGTVGLVFGDPAEISSYLIPHPVIRKVTFTGSTPVGKQLAALAGSHMKRATMELGGHAPVIVAEDADVALAVKAAGGAKFRNAGQVCISPTRFLVHNSIREEFAAALVKHAEGLKLGDGLAEGTTLGPLANARRLTAMSKVLDDARKTGAKVETGGERVGSEGNFFAPTVLTNVSLESDVFNNEPFGPIAAIRGFDKLEEAIAEANRLPFGLAGYAFTKSFTNVHLLSQQLEVGMLWINQPATPSPEMPFGGVKDSGYGSEGGPEAMEGYLVTKAVSVMSV

SEQ ID NO: 30 - protein, 481 aa, Burkholderia sp. CCGE1003
MAISSYTDTRLLINGEWCDAASGKTIDVLNPATGQVIGTVAHAGIADLDRALEAAQRGFEAWRKVPAHERAAVMRKAAALVRERASDIGRLMTQEQGKPFAEAKIEVLAAADIIEWFADEGRRLYGRVVPSRNLAAQQLVLKEPIGPVAAFTPWNFPVNQIVRKLSAALASGCSFLVKAPEETPASPAGLLQAFVEAGVPAGTVGLVFGDPAEISGYLIPHPVIRKVTFTGSTPVGKQLAALAGAHMKRATMELGGHAPVIVADDADVALAVKAAGGAKFRNAGQVCISPTRFLVHNSIREEFAAALVKHAESLKLGDGLAEGTTLGPLANARRLTAMSKVLEDARKTGAKVETGGERVGSEGNFFAPTVLTNVSLEADVFNNEPFGPIAAIRGFDKLEEAIAEANRLPFGLAGYAFTKSFSNVHLLSQQLEVGMLWINQPATPTPEMPFGGVKDSGYGSEGGPEAMEAYLVTKAVTVMSS

SEQ ID NO: 31 - protein, 481 aa, Burkholderia graminis
MAISSYTDTRLLINGEWCDAASGKTLDVLNPATGQVIGKVAHAGIADLDRALEAAQRGFEAWRKVPAHERAAVMRKAAALVRERASDIGRLMTQEQGKPFAEAKVEVLAAADIIEWFADEGRRLYGRVVPSRNLAAQQLVLKEPIGPVAAFTPWNFPVNQIVRKLSAALASGCSFLVKAPEETPASPAGLLQAFVEAGVPAGTVGLVFGDPAEISNYLIPHPVIRKVTFTGSTPVGKQLASLAGAHMKRATMELGGHAPVIVAEDADVALAVKAAGGAKFRNAGQVCISPTRFLVHNSIREEFAAALVKHAEGLKLGDGLADGTTLGPLANARRLTAMSKVLDDARRTGAKIETGGERVGTEGNFFAPTVLTNVSLEADVFNNEPFGPIAAIRGFDKLEEAIAEANRLPFGLAGYAFTKSFANVHLLSQQLEVGMLWINQPATPTPEMPFGGVKDSGYGSEGGPEAMEAYLVTKAVTVMSS

SEQ ID NO: 32 - protein, 482 aa, Burkholderia phymatum
MVTSSSYTDTRLLINNEWCDAASGKTLDVVNPATGKPIGKVAHAGKADLDRALEAAQKGFEAWRKVPANERATTMRKAAGFVRERADHIARLMTQEQGKPFAEARIEVLSAADIIEWFADEGRRVYGRVVPSRNLNAQSLVIKEPIGPVAAFTPWNFPVNQVVRKLSAALASGCSFLVKAPEETPASPAQLLQAFVDAGVPAGTVGLVFGDPAEISSYLIPHPVIRKVTFTGSTPVGKQLAALAGSHMKRATMELGGHAPVIVAEDADVALAVKAAGGAKFRNAGQVCISPTRFLVHNSIREAFAAALVKHAEGLKVGDGLAEGTQLGPLANARRLTAMASIIDNARSTGATVATGGERIGSEGNFFAPTVLTDVPLEADVFNNEPFGPIAAIRGFDNIEDAIAEANRLPFGLAGYAFTKSFRNVHLLSQNLEVGMLWINQPATPTPEMPFGGVKDSGYGSEGGPEAMEAYLVTKAVTVMAV

SEQ ID NO: 33 - protein, 480 aa, Burkholderia sp. CCGE1001
MAISSYTDTRLLINGEWCDAASGKTIDVLNPATGQVIGKVAHAGIADLDRALEAAQRGFEAWRKVPAHERAAVMRKAAALVRERASDIGRLMTQEQGKPFAEAKIEVLAAADIIEWFADEGRRLYGRVVPSRNLAAQQLVLKEPIGPVAAFTPWNFPVNQIVRKLSAALASGCSFLVKAPEETPASPAGLLQAFVEAGVPAGTVGLVFGDPAEISSYLIPHPVIRKVTFTGSTPVGKQLAALAGAHMKRATMELGGHAPVIVAEDADVALAVKAAGGAKFRNAGQVCISPTRFLVHNSIREEFAAALVKHAESLKLGDGLAEGTTLGPLANARRLTAMSKVLDDARKTGAKIETGGERVGSEGNFFAPTVLTNVSLEADVFNNEPFGPIAAIRGFDKLEEAIAEANRLPFGLAGYAFTKSFSNVHLLSQQLEVGMLWINQPATPTPEMPFGGVKDSGYGSEGGPEAMEAYLVTKAVTVMS

SEQ ID NO: 34 - protein, 481 aa, Burkholderia gladioli
MTNTTYTDTQLLINGEWCDAESGKTIDVLNPATGKVIGKVAHAGIADLDRALEAAQRGFETWRKVTAYDRAALMRKAAALVRERADTIAQLMTQEQGKPLVEAKIEVLSAADIIEWFADEGRRVYGRIVPPRNLAVQQTVVKEPVGPVAAFTPWNFPVNQVVRKLSAALATGCSFLVKAPEETPASPAQLLRAFVDAGVPAGVVGLVYGDPAEISNYLIPHPVIRKVTFTGSTPVGKQLAALAGQHMKRATMELGGHAPVIVAEDADLDLAVKAAGGAKFRNAGQVCISPTRFLVHNSVREDFAKALVKHAEGLKVGDGLALGTNLGPLANSRRLGAMEKVVADARKTGATVATGGERIGSEGNFFAPTVLTDVPLEADVFNNEPFGPIAAIRGFDSLEDAITEANRLPYGLAGYAFTRAFKNVHLLTQRLEVGMLWINQPATPWPEMPFGGVKDSGYGSEGGPEALEPYLVTKSVTVTAA

SEQ ID NO: 35 - protein, 481 aa, Burkholderia glumae
MTNTNYTDTQLLINGEWCDAASGKTLDVVNPATGQVIGKVAHAGIADLDRALDAAQRGFETWRKVSAYERSALMRKAAALVRERANSIAQLMTLEQGKPLAEARIEVLSAADIIEWFADEGRRVYGRIVPPRNLAVQQTVVKEPVGPVAAFTPWNFPVNQVVRKLSAALATGCSFLVKAPEETPASPAQLLRAFVDAGVPAGVVGLVYGDPAEISNYLIPHPVIRKITFTGSTPVGKQLAALAGQHMKRATMELGGHAPVIVAEDADLELAVKAAGGAKFRNAGQVCISPTRFLVHNSVREAFVKALVKHAEGLKVGDGLEAGTSLGPLANPRRLTAMEKVVADARKAGATVATGGERIGSAGNFFAPTVLADVPLDADVFNNEPFGPVAAVRGFDSLDDAITEANRLPYGLAGYAFTRSFKNVHLLTQRVEVGMLWINQPATPWPEMPFGGVKDSGYGSEGGPEALEPYLVTKSVTVAAV

SEQ ID NO: 36 - protein, 453 aa, Burkholderia cenocepacia
MNPATGKPIGKVAHAGIADLDRALAAAQRGFEAWRKVPAHERAATMRKAAALVRERADAIAQLMTQEQGKPLTEARVEVLSAADIIEWFADEGRRVYGRIVPPRNLNAQQTVVKEPVGPVAAFTPWNFPVNQVVRKLSAALATGCSFLVKAPEETPASPAALLRAFVDAGVPAGVIGLVFGDPAEISSYLIPHPVIRKVTFTGSTPVGKQLAALAGQHMKRATMELGGHAPVIVAEDADVALAVKAAGGAKFRNAGQVCISPTRFLVHNSIRDEFTRALVKHAEGLKVGNGLEEGTTLGALANPRRLTAMASVVDNARKVGASIETGGERIGAEGNFFAPTVIANVPLEADVFNNEPFGPVAAIRGFDKLEDAIAEANRLPFGLAGYAFTRSFANVHLLTQRLEVGMLWINQPATPWPEMPFGGVKDSGYGSEGGPEALEPYLVTKSVTVMAV

SEQ ID NO: 37 - protein, 481 aa, Burkholderia ubonensis
MAHVTYTDTQLLINGEWTDAASGKTIDVVNPATGKAIGKVAHAGIADLDRALAAAQRGFEQWRRVPAHERAATMRKAAALVRERADGIAQLMTQEQGKPLVEARLEVLAAADIIEWFADEGRRVYGRIVPPRNLGAQQTVVKEPVGPVAAFTPWNFPVNQVVRKLSAALATGCSFLVKAPEETPASPAALLRAFVDAGVPAGVIGLVYGDPAEISAYLIPHPVIRKVTFTGSTPVGKHLAALAGQHMKRATMELGGHAPVIVAEDADVALAVKAAGGAKFRNAGQVCISPTRFLVHNSIRDEFTRALVKHAQGLKVGNGLDEGTTLGALANPRRIAAMTSVVENARAVGARVETGGERIGTEGNFFAPTVLADVPLEADVFNNEPFGPVAAIRGFDSLDDAISEANRLPYGLAGYAFTRSFANVHLLTQRLEVGMLWINQPATPWPEMPFGGVKDSGYGSEGGPEALEPYLVTKSVTVMAV

SEQ ID NO: 38 - protein, 481 aa, Burkholderia sp. 383
MANVTYTDTQLLIDGEWVDAASGKTIDVVNPATGKPIGKVAHAGIADLDRALAAAQRGFDAWRKVPAHERAATMRKAAALVRERADAIAQLMTQEQGKPLTEARVEVLSAADIIEWFADEGRRVYGRIVPPRNLGAQQTVVKEPVGPVAAFTPWNFPVNQVVRKLSAALATGCSFLVKAPEETPASPAALLRAFVDAGVPAGVIGLVYGDPAEISSYLIPHPVIRKVTFTGSTPVGKQLAAMAGLHMKRATMELGGHAPVIVAEDADVALAVKAAGGAKFRNAGQVCISPTRFLVHNSIRDEFTRALVKHAEGLKVGNGLEEGTALGALANPRRLTAMASVVDNARKVGARIETGGERIGTEGNFFAPTVIADVPLEADVFNNEPFGPVAAIRGFDKLDDAIAEANRLPFGLAGYAFTRSFANVHLLTQRLEVGMLWINQPATPWPEMPFGGVKDSGYGSEGGPEALEPYLVTKSVTVMAV

SEQ ID NO: 39 - protein, 483 aa, Burkholderia dolosa
MWMANVTYTDTQLLIDGEWVDAASGKTIDVVNPATGKAIGKVAHAGIADLDRALAAAQRGFEAWRKVPAHERAATMRKAAALVRERADTIAQLMTQEQGKPLAESRIEVLSAADIIEWFADEGRRVYGRIVPPRNLGAQQTVVKEPVGPVAAFTPWNFPVNQVVRKLSAALATGCSFLVKAPEETPASPAALLRAFVDAGVPAGVIGLVFGDPAEISAYLIPHPVIRKVTFTGSTPVGKQLAALAGQHMKRATMELGGHAPVIVAEDADVALAVKAAGGAKFRNAGQVCISPTRFLVHNSIRDEFTRALVKHAEGLKVGNGLEEGTTLGALANPRRLTAMASVVDNARKVGARIETGGERIGSEGNFFAPTVIADVPLEADVFNNEPFGPVAAIRGFDKLDDAIAEANRLPYGLAGYAFTRSFANVHLLTQRLEVGMLWINQPATPWPEMPFGGVKDSGYGSEGGPEALEPYLVTKSVTVMAV

SEQ ID NO: 40 - protein, 481 aa, Azospirillum brasilense
MANVTYTDTQLLIDGEWVDAASGKTIDVVNPATGKPIGRVAHAGIADLDRALAAAQSGFEAWRKVPAHERAATMRKAAALVRERADAIAQLMTQEQGKPLTEARVEVLSAADIIEWFADEGRRVYGRIVPPRNLGAQQTVVKEPVGPVAAFTPWNFPVNQVVRKLSAALATGCSFLVKAPEETPASPAALLRAFVDAGVPAGVIGLVYGDPAEISSYLIPHPVIRKVTFTGSTPVGKQLASLAGLHMKRATMELGGHAPVIVAEDADVALAVKAAGGAKFRNAGQVCISPTRFLVHNSIRDEFTRALVKHAEGLKVGNGLEEGTTLGALANPRRLTAMASVIDNARKVGASIETGGERIGSEGNFFAPTVIANVPLDADVFNNEPFGPVAAIRGFDKLEEAIAEANRLPFGLAGYAFTRSFANVHLLTQRLEVGMLWINQPATPWPEMPFGGVKDSGYGSEGGPEALEPYLVTKSVTVMAV

SEQ ID NO: 41 - protein, 481 aa, Burkholderia multivorans
MANVTYTDTQLLIDGEWVDAASGKTIDVVNPATGRVIGKVAHAGIADLDRALAAAQRGFEAWRKVPAHERAATMRKAAALVRERADTIAQLMTQEQGKPLTEARIEVLSAADIIEWFADEGRRVYGRIVPPRNLGAQQTVVKEPVGPVAAFTPWNFPVNQVVRKLSAALATGCSFLVKAPEETPASPAALLRAFVDAGVPAGVIGLVYGDPAEISSYVIPHPVIRKVTFTGSTPVGKQLAALAGQNMKRATMELGGHAPVIVAEDADVALAVKAAGGAKFRNAGQVCISPTRFLVHNSIRDEFTRALVKHAEGLKVGNGLEEGTTLGALANPRRLTAMASVVENARKVGASVETGGERIGSEGNFFAPTVLANVPLEADVFNNEPFGPVAAIRGFDKLEDAIAEANRLPYGLAGYAFTRSFANVHLLTQRLEVGMLWINQPATPWPEMPFGGVKDSGYGSEGGPEALEPYLVTKSVTVMAV

SEQ ID NO: 42 - protein, 481 aa, Burkholderia cenocepacia
MANVTYTDTQLLIDGEWVDAASGKTIDVVNPATGKPIGKVAHASIADLDRALAAAQRGFEAWRKVPAHERAATMRKAAALVRERADTIAQLMTQEQGKPLTEARVEVLSAADIIEWFADEGRRVYGRIVPPRNLGAQQTVVKEPVGPVAAFTPWNFPVNQVVRKLSAALATGCSFLVKAPEETPASPAALLRAFVDAGVPAGVIGLVFGDPAEISSYLIPHPVIRKVTFTGSTPVGKQLAALAGQHMKRATMELGGHAPVIVAEDADVALAVKAAGGAKFRNAGQVCISPTRFLVHNSIRDEFTRALVKHAEGLKVGNGLEEGTTLGALANPRRLTAMASVVDNARKVGASIETGGERIGAEGNFFAPTVIANVPLEADVFNNEPFGPVAAIRGFDKLEDAIAEANRLPFGLAGYAFTRSFANVHLLTQRLEVGMLWINQPATPWPEMPFGGVKDSGYGSEGGPEALEPYLVTKSVTVMAV

SEQ ID NO: 43 - protein, 481 aa, Burkholderia multivorans
MANVTYTDTQLLIDGEWVDAASGKTIDVVNPATGKVIGKVAHAGIADLDRALAAAQRGFEAWRKVPAHERAATMRKAAARVRERADTIAQLMTQEQGKPLTEARIEVLSAADIIEWFADEGRRVYGRIVPPRNLGAQQTVVKEPVGPVAAFTPWNFPVNQVVRKLSAALATGCSFLVKAPEETPASPAALLRAFVDAGVPAGVIGLVYGDPAEISSYVIPHPVIRKVTFTGSTPVGKQLAALAGQHMKRATMELGGHAPVIVAEDADVALAVKAAGGAKFRNAGQVCISPTRFLVHNSIRDEFTRALVKHAEGLKVGNGLEEGTTLGALANPRRLTAMASVVENARKVGASVETGGERIGSEGNFFAPTVLANVPLEADVFNNEPFGPVAAIRGFDKLEDAIAEANRLPYGLAGYAFTRSFANVHLLTQRLEVGMLWINQPATPWPEMPFGGVKDSGYGSEGGPEALEPYLVTKSVTVMAV

SEQ ID NO: 44 - protein, 481 aa, Burkholderia sp. TJI49
MANVTYTDTQLLIDGEWVDAASGKTIDVMNPATGKVIGKVAHAGIADLDRALAAAQRGFEAWRKVPAHERAATMRKAAALVRERADAIAQLMTQEQGKPLAEARIEVLSAADIIEWFADEGRRVYGRIVPPRNLGAQQTVVKEPVGPVAAFTPWNFPVNQVVRKLSAALATGCSFLVKAPEETPASPAALLRAFVDAGVPAGVIGLVYGDPAEISSYLIPHPVIRKVTFTGSTPVGKQLAALAGQHMKRATMELGGHAPVIVAEDADVALAVKAAGGAKFRNAGQVCISPTRFLVHNSIRDEFTRALVKHAEGLKVGNGLEEGTTLGALANPRRLTAMASVVDNARKVGASVETGGERIGSEGNFFAPTVLANVPLEADVFNNEPFGPVAAIRGFDKLEDAIAEANRLPYGLAGYAFTRSFANVHLLTQRLEVGMLWINQPATPWPEMPFGGVKDSGYGSEGGPEALEPYLVTKSVTVMAV

SEQ ID NO: 45 - protein, 481 aa, Burkholderia cenocepacia
MANVTYTDTQLLIDGEWVDAASGKTIDVVNPATGKPIGKVAHAGIADLDRALAAAQRGFEAWRKVPAHERAATMRKAAALVRERADTIAQLMTQEQGKPLTEARVEVLSAADIIEWFADEGRRVYGRIVPPRNLGAQQTVVKEPVGPVAAFTPWNFPVNQVVRKLSAALATGCSFLVKAPEETPASPAALLRAFVDAGVPAGVIGLVYGDPAEISSYLIPHPVIRKVTFTGSTPVGKQLAALAGQHMKRATMELGGHAPVIVAEDADVALAVKAAGGAKFRNAGQVCISPTRFLVHNSIRDEFTRALVKHAEGLKVGNGLEEGTTLGALANPRRLTAMASVVDNARKVGASIETGGERIGAEGNFFAPTVIANVPLEADVFNNEPFGPVAAIRGFDKLEEAIAEANRLPFGLAGYAFTRSFANVHLLTQRLEVGMLWINQPATPWPEMPFGGVKDSGYGSEGGPEALEPYLVTKSVTVMAV

SEQ ID NO: 46 - protein, 481 aa, Burkholderia multivorans
MANVTYTDTQLLIDGEWVDAASGKTIDVVNPATGKVIGKVAHAGIADLDRALAAAQRGFEAWRKVPAHERAATMRKAAALVRERADTIAQLMTQEQGKPLTEARIEVLSAADIIEWFADEGRRVYGRIVPPRNLGAQQTVVKEPVGPVAAFTPWNFPVNQVVRKLSAALATGCSFLVKAPEETPASPAALLRAFVDAGVPAGVIGLVYGDPAEISSYVIPHPVIRKVTFTGSTPVGKQLAALAGQHMKRATMELGGHAPVIVAEDADVALAVKAAGGAKFRNAGQVCISPTRFLVHNSIRDEFTRALVKHAEGLKVGNGLEEGTTLGALANPRRLTAMASVVENARKVGASVETGGERIGSEGNFFAPTVLANVPLEADVFNNEPFGPVAAIRGFDKLEDAIAEANRLPYGLAGYAFTRSFANVHLLTQRLEVGMLWINQPATPWPEMPFGGVKDSGYGSEGGPEALEPYLVTKSVTVMAV

SEQ ID NO: 47 - protein, 481 aa, Burkholderia vietnamiensis
MANVTYTDTQLLIDGEWVDAASGKTIDVVNPATGKAIGKVAHAGIADLDRALAAAQRGFEAWRKVPAHERAATMRKAAALVRERADAIAQLMTQEQGKPLTEARIEVLSAADIIEWFADEGRRVYGRIVPPRNLGAQQMVVKEPVGPVAAFTPWNFPVNQVVRKLCAALATGCSFLVKAPEETPASPAALLRAFVDAGVPAGVVGLVYGDPAEISSYLIPHPVIRKVTFTGSTPVGKQLAALAGQHMKRATMELGGHAPVIVAEDADVALAVKAAGGAKFRNAGQVCISPTRFLVHNSIRDEFTRALVAHAQGLKIGNGLDEGTTLGALANPRRLTAMASVVENARKVGASIETGGERIGSEGNFFAPTVIANVPLEADVFNNEPFGPVAAIRGFDKLEDAISEANRLPFGLAGYAFTRSFANVHLLTQRLEVGMLWINQPATPWPEMPFGGVKDSGYGSEGGPEALEPYLVTKSVTVMAV

SEQ ID NO: 48 - protein, 481 aa, Burkholderia cenocepacia
MANVTYTDTQLLIDGEWVDAASGKTIDVVNPATGKPIGKVAHAGIADLDRALAAVQRGFEAWRKVPAHERAATMRKAAALVRERADTIAQLMTQEQGKPLTEARVEVLSAADIIEWFADEGRRVYGRIVPPRNFNAQQTVVKEPVGPVAAFTPWNFPVNQVVRKLSAALATGCSFLVKAPEETPASPAALLRAFVDAGVPAGVIGLVFGDPAEISSYLIPHPVIRKVTFTGSTPVGKQLAALAGQHMKRATMELGGHAPVIVAEDADVALAVKAAGGAKFRNAGQVCISPTRFLVHNSIRDEFTRALVKHAEGLKVGNGLEEGTTLGALANPRRLTAMASVVDNARKVGASIETGGERIGAEGNFFAPTVIANVPLEADVFNNEPFGPVAAIRGFDKLEDAIAEANRLPFGLAGYAFTRSFANVHLLTQRLEVGMLWINQPATPWPEMPFGGVKDSGYGSEGGPEALEPYLVTKSVTVMAV

SEQ ID NO: 49 - protein, 481 aa, Burkholderia ambifaria
MANVTYTDTQLLIDGEWVDAASGKTIDVVNPATGKAIGKVAHAGIADLDRALAAAQRGFEAWRKVPANERAATMRKAAALVRERADTIAQLMTQEQGKPLTEARVEVLSAADIIEWFADEGRRVYGRIVPPRNLGAQQMVVKEPVGPVAAFTPWNFPVNQVVRKLSAALATGCSFLVKAPEETPASPAALLRAFVDAGVPAGVIGLVYGDPAEISSYLIAHPVIRKVTFTGSTPVGKQLAALAGQHMKRATMELGGHAPVIVAEDADVALAVKAAGGAKFRNAGQVCISPTRFLVHNSIRDEFTRALVQHAEGLKIGNGLEEGTTLGALANPRRLTAMVSVVDNARKVGARIETGGERIGSEGNFFAPTVIANVPLEADVFNNEPFGPVAAIRGFDKLDDAIAEANRLPFGLAGYAFTRSFANVHLLTQRLEVGMLWINQPATPWPEMPFGGVKDSGYGSEGGPEALEPYLVTKSVTVMAV

SEQ ID NO: 50 - protein, 481 aa, Burkholderia ambifaria
MANVTYTDTQLLIDGEWVDAASGKTIDVVNPATGKAIGKVAHAGIADLDRALVAAQRGFEAWRKVPANERAATMRKAAALVRERADTIAQLMTQEQGKPLTEARVEVLSAADIIEWFADEGRRVYGRIVPPRNLGAQQMVVKEPVGPVAAFTPWNFPVNQVVRKLSAALATGCSFLVKAPEETPASPAALLRAFVDAGVPAGVIGLVYGDPAEISSYLIAHPVIRKVTFTGSTPVGKQLAALAGQHMKRATMELGGHAPVIVAEDADVALAVKAAGGAKFRNAGQVCISPTRFLVHNSIRDEFTRALVQHAEGLKIGNGLEEGTTLGALANPRRLTAMASVVDNARKVGASIETGGERIGSEGNFFAPTVIANVPLEADVFNNEPFGPVAAIRGFDKLEDAIAEANRLPFGLAGYAFTRSFANVHLLTQRLEVGMLWINQPATPWPEMPFGGVKDSGYGSEGGPEALEPYLVTKSVTVMAV

SEQ ID NO: 51 - protein, 481 aa, Burkholderia ambifaria
MANVTYTDTQLLIDGEWVDAASGRTIDVVNPATGKAIGKVAHAGIADLDRALAAAQRGFEAWRKVPANERAATMRKAAALVRERADAIAQLMTQEQGKPLTEARVEVLSAADIIEWFADEGRRVYGRIVPPRNLGAQQMVVKEPVGPVAAFTPWNFPVNQVVRKLSAALATGCSFLVKAPEETPASPAALLRAFVDAGVPAGVIGLVYGDPAEISSYLIAHPVIRKVTFTGSTPVGKQLAALAGQHMKRATMELGGHAPVIVAEDADVALAVKAAGGAKFRNAGQVCISPTRFLVHNSIRDEFTRALVQHAEGLKIGNGLEEGTTLGALANPRRLTAMASVVENARKVGASIETGGERIGSEGNFFAPTVIANVPLEADVFNNEPFGPVAAIRGFDKLEDAIAEANRLPFGLAGYAFTRSFANVHLLTQRLEVGMLWINQPATPWPEMPFGGVKDSGYGSEGGPEALEPYLVTKSVTVMAV

SEQ ID NO: 52 - protein, 481 aa, Burkholderia ambifaria
MANVTYTDTQLLIDGEWVDAASGKTIDVVNPATGKAIGKVAHAGIADLDRALAAAQRGFEAWRKVPANERAATMRKAAALVRERADAIAQLMTQEQGKPLTEARVEVLSAADIIEWFADEGRRVYGRIVPPRNLGAQQMVVKEPVGPVAAFTPWNFPVNQVVRKLSAALATGCSFLVKAPEETPASPAALLRAFVDAGVPAGVIGLVYGEPAEISSYLIAHPVIRKVTFTGSTPVGKQLAALAGQHMKRATMELGGHAPVIVAEDADVALAVKAAGGAKFRNAGQVCISPTRFLVHNSIRDEFTRALVQHAEGLKIGNGLEEGTTLGALANPRRLTAMASVVENARKVGASIETGGERIGSEGNFFAPTVIANVPLEADVFNNEPFGPVAAIRGFDKLEDAIAEANRLPFGLAGYAFTRSFANVHLLSQRLEVGMLWINQPATPWPEMPFGGVKDSGYGSEGGPEALEPYLVTKSVTVMAV

SEQ ID NO: 53 - protein, 500 aa, Saccharomyces cerevisiae
MTKLHFDTAEPVKITLPNGLTYEQPTGLFINNKFMKAQDGKTYPVEDPSTENTVCEVSSATTEDVEYAIECADRAFHDTEWATQDPRERGRLLSKLADELESQIDLVSSIEALDNGKTLALARGDVTIAINCLRDAAAYADKVNGRTINTGDGYMNFTTLEPIGVCGQIIPWNFPIMMLAWKIAPALAMGNVCILKPAAVTPLNALYFASLCKKVGIPAGVVNIVPGPGRTVGAALTNDPRIRKLAFTGSTEVGKSVAVDSSESNLKKITLELGGKSAHLVFDDANIKKTLPNLVNGIFKNAGQICSSGSRIYVQEGIYDELLAAFKAYLETEIKVGNPFDKANFQGAITNRQQFDTIMNYIDIGKKEGAKILTGGEKVGDKGYFIRPTVFYDVNEDMRIVKEEIFGPVVTVAKFKTLEEGVEMANSSEFGLGSGIETESLSTGLKVAKMLKAGTVWINTYNDFDSRVPFGGVKQSGYGREMGEEVYHAYTEVKAVRIKL

SEQ ID NO: 54 - protein, 506 aa, Saccharomyces cerevisiae
MPTLYTDIEIPQLKISLKQPLGLFINNEFCPSSDGKTIETVNPATGEPITSFQAANEKDVDKAVKAARAAFDNVWSKTSSEQRGIYLSNLLKLIEEEQDTLAALETLDAGKPYHSNAKGDLAQILQLTRYFAGSADKFDKGATIPLTFNKFAYTLKVPFGVVAQIVPWNYPLAMACWKLQGALAAGNTVIIKPAENTSLSLLYFATLIKKAGFPPGVVNIVPGYGSLVGQALASHMDIDKISFTGSTKVGGFVLEASGQSNLKDVTLECGGKSPALVFEDADLDKAIDWIAAGIFYNSGQNCTANSRVYVQSSIYDKFVEKFKETAKKEWDVAGKFDPFDEKCIVGPVISSTQYDRIKSYIERGKREEKLDMFQTSEFPIGGAKGYFIPPTIFTDVPQTSKLLQDEIFGPVVVVSKFTNYDDALKLANDTCYGLASAVFTKDVKKAHMFARDIKAGTVWINSSNDEDVTVPFGGFKMSGIGRELGQSGVDTYLQTKAVHINLSLDN

SEQ ID NO: 55 - protein, 506 aa, Saccharomyces cerevisiae
MPTLYTDIEIPQLKISLKQPLGLFINNEFCPSSDGKTIETVNPATGEPITSFQAANEKDVDKAVKAARAAFDNVWSKTSSEQRGIYLSNLLKLIEEEQDTLAALETLDAGKPFHSNAKQDLAQIIELTRYYAGAVDKFNMGETIPLTFNKFAYTLKVPFGVVAQIVPWNYPLAMACRKMQGALAAGNTVIIKPAENTSLSLLYFATLIKKAGFPPGVVNVIPGYGSVVGKALGTHMDIDKISFTGSTKVGGSVLEASGQSNLKDITLECGGKSPALVFEDADLDKAIEWVANGIFFNSGQICTANSRVYVQSSIYDKFVEKFKETAKKEWDVAGKFDPFDEKCIVGPVISSTQYDRIKSYIERGKKEEKLDMFQTSEFPIGGAKGYFIPPTIFTDVPETSKLLRDEIFGPVVVVSKFTNYDDALKLANDTCYGLASAVFTKDVKKAHMFARDIKAGTVWINQTNQEEAKVPFGGFKMSGIGRESGDTGVDNYLQIKSVHVDLSLDK